(19) United States
(12) Patent Application Publication
(10) Pub. No.: US 2023/0182041 A1
(43) Pub. Date: Jun. 15, 2023

(54) PURIFICATION OF ANTIBODIES

(71) Applicant: MEDIMMUNE, LLC, Gathersburg, MD (US)

(72) Inventors: Gisela FERREIRA, Gaithersburg, MD (US); Methal ALBARGHOUTHI, Gaithersburg, MD (US); Min ZHU, Gaithersburg, MD (US); Melani STONE, Gaithersburg, MD (US); David ROBBINS, Gaithersburg, MD (US)

(21) Appl. No.: 18/050,104

(22) Filed: Oct. 27, 2022

Related U.S. Application Data
(63) Continuation of application No. 16/393,411, filed on Apr. 24, 2019, now abandoned.
(60) Provisional application No. 62/662,331, filed on Apr. 25, 2018.

(51) Int. Cl. B01D 15/38; C07K 1/22; B01D 15/36; C07K 16/28; C07K 1/16; C07K 1/36

(52) U.S. Cl. CPC B01D 15/3809; B01D 15/362; B01D 15/363; C07K 1/22; C07K 1/36; C07K 1/165; C07K 16/2827; B01D 15/206

(57) ABSTRACT

The disclosure provides methods for the isolation, separation, and purification of antibodies. The method comprises an affinity chromatography capture step, anion exchange chromatography polishing step, and cation exchange chromatography polishing step.

### CROSS REFERNCE TO RELATED APPLICATIONS

This application is a continuation of U.S. Pat. Application Serial No. 16/393,411, filed Apr. 24, 2019, which claims priority to and the benefit of U.S. Provisional Pat. Application Serial No. 62/662,331, filed Apr. 25, 2018. The entire contents of each of these applications are hereby incorporated by reference herein.

### REFERENCE TO AN ELECTRONIC SEQUENCE LISTING

A computer readable form of the Sequence Listing is filed with this application by electronic submission and is incorporated into this application by reference in its entirety. The Sequence Listing is contained in the file created on Oct. 24, 2022 having the file name “18-122-US.xml” and is 11 kb in size.

### FIELD OF THE INVENTION

The present disclosure relates to the purification of antibodies. More specifically, the disclosure relates to methods of separating an antibody from host cell proteins, viruses, and other contaminants in the preparation of a pharmaceutical composition comprising the purified antibody.

### BACKGROUND

Monoclonal antibodies have a wide range of uses in diagnostics and therapeutics. Most of these uses require homogeneous antibody preparations. Antibodies are often expressed and isolated from plasma, serum, ascites fluid, cell culture medium, and bacterial cultures. These are all sources which contain numerous contaminants. Therefore, the efficient purification of antibodies from such sources is necessary.

The process of developing antibodies for commercial applications generally involves the expression of an antibody in a host cell followed by isolation and purification of the antibody from the host cell. Expression of the antibody generally involves culturing a prokaryotic or eukaryotic host cell under appropriate conditions for the host cells to produce the antibody.

After an antibody is expressed, intact host cells and cell debris are separated from the cell culture media in a process referred to as “cell harvesting.” For example, host cells can be separated from the cell culture media by centrifugation or filtration to provide a clarified fluid (which can be referred to as the “cell culture supernatant”) that includes the antibody and other impurities. Examples of impurities that may be found in the clarified cell culture supernatant include, but are not limited to, host cell proteins (HCP), nucleic acids, endotoxins, viruses, protein variants, and protein aggregates.

Purification refers to the removal of impurities from the clarified cell culture supernatant and typically involves one or more chromatography steps. Typical processes include capture, intermediate purification or polishing, and final polishing steps. Additionally, virus deactivation and filtration steps are included throughout the process. Affinity chromatography, such as Protein A chromatography, or ion exchange chromatography is often used as a capture step. Capture may be followed by one or more intermediate purification or polishing steps to increase purity and/or remove viral contaminants. Intermediate purification or polishing steps are often accomplished by affinity chromatography, ion exchange chromatography, or hydrophobic interaction chromatography (HIC). In many processes, the final polishing step is accomplished using ion exchange chromatography, hydrophobic interaction chromatography, or gel filtration.

In order to increase the productivity of the purification process, larger cell culture monoclonal antibody titers are becoming more common; however, the increase in cell culture monoclonal antibody titers can be problematic for downstream purification processes and lead to capture limitations. In order to combat these capture limitations, larger chromatography columns are used along with performing more cycles, which increases the space and time needed for monoclonal antibody purification processes.

In any given commercial facility, several monoclonal antibodies can be in production at one time. Processing a monoclonal antibody can require five different process flows, ten different resins, and as many as sixteen different process buffers. The footprint of the operation for each monoclonal antibody, along with the number of individual buffers required and the length and variation in time creates a serious bottleneck for the commercial facility. Hence, there remains a need for an optimized universal purification platform for monoclonal antibodies in the commercial space.

### SUMMARY

The present disclosure provides methods for the isolation and purification of antibodies.

In one aspect, the disclosure provides methods of purifying an antibody, the methods comprising: (a) subjecting a sample comprising the antibody to affinity chromatography to capture the antibody and eluting the antibody from the affinity chromatography column to produce an eluate comprising the antibody; (b) treating the eluate from step (a) by adding an amount of a one or more solutions that reduces or increases the pH of the eluate to produce a neutralized eluate comprising the antibody; (c) subjecting the neutralized eluate from step (b) to anion exchange chromatography and collecting a flow-through product comprising the antibody; (d) subjecting the flow-through product from step (c) to cation exchange chromatography and eluting the antibody from the cation exchange chromatography column to produce a cation exchange chromatography product comprising the antibody; (e) subjecting the cation exchange chromatography product from step (d) to virus filtration to produce a virus filtration product comprising the antibody; and (f) subjecting the virus filtration product from step (e) to ultrafiltration to recover the purified antibody.

In some embodiments, the affinity chromatography column is a protein-A based resin column.

In some embodiments, the protein-A based resin column is a MabSelect SuRe column.

In some embodiments, the step of subjecting the sample comprising the antibody to affinity chromatography comprises: (i) equilibrating the affinity chromatography column with an equilibrating buffer at pH 6.0 to pH 8.0; (ii) re-equilibrating the affinity chromatography column with the equilibrating buffer at pH 6.0 to pH 8.0; and (iii) eluting the antibody from the affinity chromatography column using an elution buffer at pH 3.0 to pH 4.0.

In some embodiments, the equilibrating buffer comprises 50 mM Tris at pH 7.4.

In some embodiments, the elution buffer comprises 50 mM sodium acetate at pH 3.6.

In some embodiments of the disclosure, a further step comprises washing the affinity chromatography column following step (i) and prior to step (ii).

In some embodiments, the affinity chromatography column is washed with a washing buffer comprising: (i) 1 M or lower arginine at pH 6.0 to pH 8.0; (ii) at least 45 mM sodium caprylate at pH 6.0 to pH 10; (iii) at least 45 mM sodium caprylate and, 4 M or lower sodium chloride at pH 6.0 to pH 8.0; or (iv) 4 M or lower sodium chloride at pH 6.0 to pH 8.0.

In some embodiments, the washing buffer comprises: (i) 58 mM sodium phosphate and 0.5 M arginine at pH 7.0; (ii) 100 mM Tris, 50 mM sodium caprylate, and 2.5 M sodium chloride at pH 9.0; or (iii) 52 mM Tris and 1.0 M sodium chloride at pH 7.4.

In some embodiments, the step of treating the eluate to produce a neutralized eluate comprising the antibody comprises: (i) titrating the eluate to a pH range of pH 3.0 to pH 5.0 using an acidic titrant solution; (ii) incubating the eluate and acidic titrant solution in the titrated pH range for at least 15 minutes; and (iii) neutralizing the eluate and acidic titrant solution to neutral pH using a basic titrant solution.

In some embodiments, the acidic titrant solution comprises about 100 mM glycine to about 500 mM glycine at pH 2.0 to pH 3.0 and the basic titrant solution comprises about 0.5 M Tris to about 1.0 M Tris.

In some embodiments, the acidic titrant solution comprises 248 mM glycine at pH 2.35 and the basic titrant solution comprises 0.5 M Tris.

In some embodiments, the neutralized eluate of step (b) comprises filtering the neutralized eluate of step (b) through at least one 0.2 µm filter prior to subjecting the neutralized eluate to anion exchange chromatography.

In some embodiments, the methods further comprise filtering the neutralized eluate of step (b) through a depth filter, and then through a 0.2 µm filter prior to subjecting the neutralized eluate to the anion exchange chromatography.

In some embodiments, the step of subjecting the neutralized eluate to anion exchange chromatography is performed using a resin-based chromatography membrane.

In some embodiments, the anion exchange chromatography is performed using Capto Q resin.

In some embodiments, the anion exchange chromatography is performed using Mustang Q membrane.

In some embodiments, the neutralized eluate from step (b) comprises up to 500 mg/mL of the antibody when subjected to anion exchange chromatography.

In some embodiments, the neutralized eluate from step (b) is subjected to anion exchange chromatography and collecting a flow through product comprising the antibody, which comprises: (i) equilibrating the anion exchange chromatography column with an equilibrating buffer at pH 6.0 to pH 8.0; (ii) passing the neutralized eluate through the anion exchange chromatography column; and (iii) collecting the flow through product comprising the antibody from the anion exchange chromatography column.

In some embodiments, the neutralized eluate is chased with a chase solution at pH 3.0 to pH 4.0 and collecting the chase solution that flows through the anion exchange chromatography column to produce a flow through product comprising the antibody.

In some embodiments, the cation exchange chromatography is performed using an HS resin-based column.

In some embodiments, the cation exchange chromatography is performed using an Applied Biosystems POROS Strong Cation Exchange Media 50 micron column.

In some embodiments, the step of subjecting the flow product comprising the antibody to a cation exchange chromatography comprises: (i) equilibrating the cation exchange chromatography column with a cation exchange equilibrium buffer at pH 4.5 to pH 5.5; (ii) loading up to 100 mg/mL of flow through product onto the cation exchange chromatography column; (iii) washing the cation exchange chromatography column with a cation exchange washing buffer at pH 5.0 to pH 7.0; and (iv) eluting a cation exchange chromatography product comprising the antibody from the cation exchange chromatography column using a cation exchange elution buffer at pH 5.0 to pH 7.0.

In some embodiments, the cation exchange equilibrium buffer comprises 50 mM sodium acetate at pH 5.0, the cation exchange washing buffer comprises 26 mM Histidine/Histidine-HCl at pH 5.8, and the cation exchange elution buffer comprises 20 mM Histidine/Histidine-HCl and 115 mM sodium chloride at pH 6.0.

In some embodiments, the step of subjection the cation exchange chromatography product to virus filtration is performed using a Millipore Vpro+ virus filtration membrane.

In some embodiments, the step of subjecting virus filtration product to ultrafiltration is performed using a TFF membrane.

In some embodiments, the purified antibody is a human anti-PD-L1 antibody.

In some embodiments, the human anti-PD-L1 antibody comprises a light chain region comprising the amino acid sequence of SEQ ID NO: 1 and a heavy chain region comprising the amino acid sequence of SEQ ID NO: 2.

In some embodiments, the human anti-PD-L1 antibody comprises the amino acid sequences of SEQ ID NOs: 3-8.

In some embodiments, purified antibody recovered in step (f) constitutes about 60% to about 70% of antibody in the sample comprising the antibody.

In some embodiments, at least 99% of the purified antibody recovered in step (f) is present as a monomer as measured by high pressure size exclusion chromatography (HP-SEC).

In some embodiments, the DNA content of the purified antibody recovered in step (f) is less than 0.2 pg/mg.

In some embodiments, less than about 1% purified antibody recovered in step (f) forms an aggregate as measured by high pressure size exclusion chromatography (HP-SPEC).

In some embodiments, host cell protein content of the purified antibody recovered in step (f) is less than 10 ng/mg.

In one aspect, the disclosure provides a method for producing a purified antibody preparation, the method comprising: (a) subjecting a sample comprising the antibody to affinity chromatography to capture the antibody and eluting the antibody from the affinity chromatography column to produce an eluate comprising the antibody; (b) treating the eluate from step (a) by adding an amount of one or more solutions that reduces or increases the pH of the eluate to produce a neutralized eluate comprising the antibody; (c) subjecting the neutralized eluate from step (b) to anion exchange chromatography and collecting a flow-through product comprising the antibody; (d) subjecting the flow-through product from step (c) to cation exchange chromatography and eluting the antibody from the cation exchange chromatography column to produce a cation exchange chromatography product comprising the antibody; (e) subjecting the cation exchange chromatography product from step (d) to virus filtration to produce a virus filtration product comprising the antibody; and (f) subjecting the virus filtration product from step (e) to ultrafiltration to produce the purified antibody preparation.

In some embodiments, the purified antibody preparation comprises a main form of the antibody comprising greater than, or equal to, 45% of the protein in the composition as measured using capillary isoelectric focusing (cIEF), acidic forms of the antibody comprising 45% to 50% of the protein in the composition as measured using cIEF, and a basic form of the antibody comprising 18% to 23% of the protein in the composition as measured using cIEF.

In some embodiments, 1.5% to 2.5% of the antibody in the antibody preparation forms an aggregate as determined by high-pressure size exclusion chromatography (HP-SPEC); and wherein 97% to 98% of the antibody in the antibody preparation is present as a monomer as measured by HP-SEC.

In some embodiments, the glycan structures of the purified antibody preparation comprise G0f, G1f, G2f, and G0 glycoforms. In some embodiments, the glycan structures of the purified antibody preparation have a content greater than about 90% for the G0f, G1f, G2f, and G0 forms. In some embodiments, the purified antibody preparation comprises about 71.9% G0f content, 18.4% G1f content, 1.5% G2f content, and 1.9% G0 content.

Specific embodiments of the disclosure will become evident from the following more detailed description of some embodiments and the claims. It is noted that the scope of the claims is defined by the recitations therein and not by the specific discussion of features and advantages set forth in the present description.

### BRIEF DESCRIPTIONS OF THE DRAWINGS

FIG. 1 shows a schematic of the process steps in the antibody purification process of the disclosure.

FIGS. 2A and 2B show equilibrium binding capacity (EBC) and dynamic binding capacity (DBC) comparisons of the monoclonal antibody MEDI-7814, which was purified using MabSelect Sure LX, MabSelect SuRe, MabCapture A, and ProSep Ultra Plus affinity chromatography resins.

FIG. 3A shows the dynamic binding capacity of three monoclonal antibodies (MEDI-3617, MEDI-573, and MEDI-7814) captured by ProSep Ultra Plus (PUP) and MabSelect SuRe (MSS) affinity chromatography.

FIG. 3B shows the dynamic binding capacity for MEDI-7814 at 10% breakthrough using both ProSep Ultra Plus and MabSelect SuRe affinity chromatography.

FIG. 4 shows the loading safety factor through the use of breakthrough curve comparison of MEDI-7814 captured through either ProSep Ultra Plus (PUP) or MabSelect SuRe (MSS) affinity chromatography.

FIG. 5 shows a comparison of the amount of host cell protein in the eluate for three monoclonal antibodies (MED-3617, MEDI-573, and MEDI-7814) captured through either ProSep Ultra Plus (PUP) or MabSelect SuRe (MSS) affinity chromatography. The measurements for MEDI-573 are from a first-generation capture trial using either ProSep Ultra Plus or MabSelect SuRe affinity chromatography.

FIG. 6A shows a comparison of elution volume for two monoclonal antibodies (MEDI-3617 and MEDI-573) captured using either ProSep Ultra Plus (PUP) or MabSelect SuRe (MSS) affinity chromatography.

FIG. 6B shows the elution profiles for MEDI-573 after the capture step using either ProSep Ultra Plus or MabSelect SuRe affinity chromatography.

FIG. 7 shows a comparison of the percent yield for two monoclonal antibodies (MEDI-3617 and MEDI-573) as measured after the capture step using an antibody purification process of the disclosure, wherein the capture step was performed using either ProSep Ultra Plus (PUP) and MabSelect SuRe (MSS) affinity chromatography.

FIG. 8A shows the response of ProSep Ultra Plus media to increased flow rate.

FIG. 8B shows the pressure/flow profile for MabSelect SuRe in a packed bed (bed height 20 cm) in an AxiChrom 300 column (internal diameter 300 mm).

FIG. 9A shows acidic titrant evaluation using 1 M acetic acid, pH 2.38 as the acidic titrant for low pH viral inactivation during purification of the monoclonal antibody durvalumab.

FIG. 9B shows acidic titrant evaluation using 100 mM glycine, pH 2.35 as the acidic titrant for low pH viral inactivation during purification of durvalumab.

FIGS. 10A-10B show neutralization of low pH viral inactivated eluate after acidification using 1 M acetic acid, pH 2.38 during purification of the monoclonal antibody durvalumab. FIG. 10A shows neutralization using 1 M Tris, pH 11, and FIG. 10B shows neutralization using 250 mM phosphate, pH 9 during purification of durvalumab.

FIGS. 11A-11B show neutralization of low pH viral inactivated eluate after acidification using 100 mM glycine, pH 2.35 during purification of the monoclonal antibody durvalumab. FIG. 11A shows neutralization using 1 M Tris, pH 11, and FIG. 11B shows neutralization using 250 mM phosphate, pH 9 during purification of durvalumab.

FIG. 12 shows the titration curve of 250 mM glycine, pH 2.35 during low pH treatment in the purification of the monoclonal antibody durvalumab. The ratio of acid to load was 0.12 mL acid/mL load to reach a target pH of 3.5.

FIG. 13 shows the titration curves for 0.5 M and 1 M Tris during neutralization following low pH treatment in the purification of the monoclonal antibody durvalumab. The ratio of base to load was 0.11 mL base/mL load to reach a target pH of 7.5.

FIG. 14 shows the breakthrough curves for host cell protein and DNA for Mustang Q membrane anion exchange chromatography purification of durvalumab.

FIG. 15 shows the protein load and dynamic binding capacities for the purification of the monoclonal antibody durvalumab after Poros HS50 cation exchange chromatography.

FIG. 16 shows the Poros HS 50 gradient elution profile for the monoclonal antibody durvalumab, including the percentage eluted as a monomer.

FIG. 17 shows the results of an HS 50 fractionation experiment in which eluate fractions of the monoclonal antibody durvalumab were collected in the ranges: main peak up to 2.5 OD (500 mAU), 2.5 to 2.0 OD (500 mAU to 400 mAU), 2.0 to 1.0 OD, and 1.0 to 0.5 OD.

FIG. 18 shows the host cell protein and DNA concentrations as measured from the eluate of the durvalumab HS 50 fractionation experiment.

FIG. 19 shows the results of a clean run after anion exchange chromatography of durvalumab by way of a chromatogram.

FIG. 20 shows a chromatogram of the analysis after anion exchange chromatography of a durvalumab bench clean run.

FIG. 21 shows a comparison of feed pressure versus bulk concentration for four filtration membranes: Hydrosart ECO, Hydrosart, Pall Omega T, and Pellicon 3 Ultracell.

FIG. 22 demonstrates Ultra Performance Liquid Chromatography (UPLC) peak identification of 2-AB labeled oligosaccharides on durvalumab.

FIG. 23 shows a Differential Scanning Calorimetry (DSC) profile of durvalumab at in formulation buffer wherein Tm₁ was demonstrated to be 64.5° C. and Tm₂ was demonstrated to be 73.04° C.

FIG. 24 is a capillary Isoelectric focusing (cIEF) profile of durvalumab in formulation buffer. The four peaks displayed a pI range between 8.3 and 8.8. The main peak had a pI of 8.6.

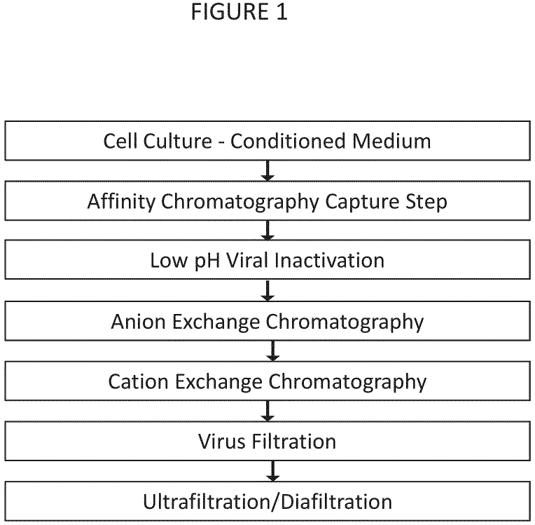

### DETAILED DESCRIPTION

The disclosure provides methods for purification of antibodies.

As utilized in accordance with the present disclosure, the following terms, unless otherwise indicated, shall be understood to have the following meanings. Unless otherwise required by context, singular terms shall include pluralities and plural terms shall include the singular.

The term “antibody” refers to a molecule comprising at least one binding domain of a given antigen and a constant domain comprising an Fc fragment capable of binding to Fc receptors (FcR). In most mammals, such as humans and mice, an antibody is composed of four polypeptide chains: two heavy chains and two light chains linked together by a variable number of disulfide bridges that provide the molecule with flexibility. Each light chain consists of a constant domain (CL) and a variable domain (VL); the heavy chains being composed of a variable domain (VH) and three or four constant domains (CH1 to CH3 or CH1 to CH4) depending on the antibody isotype. In a few rare mammals, such as camels and llamas, antibodies consist of only two heavy chains, each heavy chain comprising a variable domain (VH) and a constant region.

“Host cell proteins” (HCPs) are low-level, process-related protein impurities in products derived from the host organism during biotherapeutic manufacturing. During expression of an antibody, host cell systems can express many endogenous proteins. HCP can be seen in high amounts (sometimes > than 1,000 ,000 ng/mg) in harvested cell culture fluid. Purification of the antibody from such HCP contaminants can be challenging, with low-level contamination remaining after purification. HCPs accompanied with recombinant biotherapeutics can significantly affect drug efficacy and cause immunogenicity. Methods for determining the residual levels of host cell protein (HCP) concentration are known and include, for example, detecting residual HCP levels using an immunoassay, such as an enzyme-linked immunosorbent assay (ELISA), 1D and 2D sodium dodecyl sulfate-polyacrylamide gel electrophoresis (SDS-PAGE), 2D-differential in-gel electrophoresis (DIGE), capillary zone electrophoresis-electrospray ionization-tandem mass spectrometry (CZE-ESI-MS/MS), or two-dimensional-liquid chromatography-tandem mass spectrometry (2D-LC-MS/MS). In ELISA, for example, the primary antibody is specific to the HCPs produced from a particular host cell, e.g., CHO cells or E. coli cells, used to generate the recombinant polypeptide.

The term “recombinant” refers to a biological material, for example, a nucleic acid or protein, that has been artificially or synthetically (i.e., non-naturally) altered by human intervention.

The terms “stability” and “stable,” as used herein in the context of a formulation of an antibody or antibody fragment, refer to the resistance of the antibody under manufacture, preparation, transportation, and storage conditions. A “stable” formulation retains biological activity under manufacture, preparation, transportation, and storage conditions. Stability can be assessed by degrees of particle formation, aggregation, degradation, or fragmentation, as measured by high pressure size exclusion chromatography (HPSEC), static light scattering (SLS), Fourier transform infrared spectroscopy (FTIR), circular dichroism (CD), urea unfolding techniques, intrinsic tryptophan fluorescence, differential scanning calorimetry, and/or ANS binding techniques, as compared to a reference formulation.

### 1. Harvest

In the production of antibodies for pharmaceutical purposes, antibodies are expressed in mammalian host cells and grown in suspension culture in large bioreactors. The majority of commercial monoclonal antibodies are derived from just a few host cell lines, including Chinese Hamster Ovary (CHO), NS0, Sp2/0, with CHO being the popular choice. In order to prepare the antibody for purification, intact host cells and host cell debris from the culture media is removed through harvesting. Harvesting is generally accomplished by centrifugation, flocculation/precipitation, depth filtration, and sterile filtration, although other approaches can be used. Harvesting yields a solution, or sample, comprising the antibody.

### 2. Capture

The methods disclosed herein comprise capturing an antibody on an affinity chromatography column and eluting the antibody from the affinity chromatography column to produce an eluate. Affinity chromatography refers to a chromatographic method of separating biochemical mixtures based on the specific and reversible interaction between a biomolecule such as an antibody, to a specific binding partner covalently coupled to the solid phase. In some embodiments, affinity chromatography involves the use of microbial proteins, such as protein A, protein G, protein A/G, or protein L. Protein A is a bacterial cell wall protein that binds to mammalian IgGs primarily through their Fc regions. Protein A resin is useful for affinity purification and isolation of a variety antibody isotypes, particularly IgG1, IgG2, and IgG4. There are many protein A resins available that are suitable for use in the purification process described herein, such as MabSelect SuRe LX (GE Healthcare), MabSelect SuRe (GE Healthcare), Protein A Sepharose 4 Fast Flow (GE Healthcare), Provance Protein A (Grace & Co.), Toyopearl AF-rProtein A HC (Tosoh Bioscience), ProSep Ultra Plus (Millipore Sigma), Poros MabCapture A (Applied Biosystems), and AbSolute High Cap (AGC Si-Tech). The resins are generally classified based on their backbone composition and include, for example, glass or silica-based resins, agarose-based resins, and organic polymer based resins. In some embodiments of the methods disclosed herein, the affinity chromatography column is a protein A based resin column used to capture the antibody. In some embodiments, the protein A based resin column is a MabSelect SuRe column.

The term “flow rate” refers to the volume of liquid phase passed through the chromatography column or over the membrane in unit time. The flow rate through an affinity chromatography column is an important parameter for optimizing separation. Although a reduced separation time may be desirable, a flow rate that is too fast may cause the mobile phase to move past the solid phase faster than the diffusion time necessary to reach the internal bead volume. Generally, a flow rate of at least about 50 cm/hour, 100 cm/hour, 150 cm/hour, 200 cm/hour, or 250 cm/hour and up to about 300 cm/hour, 350 cm/hour, 400 cm/hour, 450 cm/hour, or 500 cm/hour is used. In some embodiments, the solution comprising the antibody is loaded onto the affinity chromatography column at a flow rate of at least about 100 cm/hour to about 200 cm/hour. In some embodiments, the passing of the liquid phase through the solid phase comprises more than one flow rate. For example, the flow rate for loading the column starts at a higher flow rate, and once a certain threshold is reached, i.e., the concentration of target antibody, the flow rate is decreased until column capacity is reached. In some embodiments, the solution comprising the antibody is loaded onto the affinity chromatography column at about 300 cm/hour, 350 cm/hour, or 400 cm/hour until about 60-95% column capacity is reached, at which time the flow rate is decreased to a rate of at least about 50 cm/hour, 100 cm/hour, or 150 cm/hour. The column dimensions can also be varied where large scale or commercial production scales using columns having diameters of up to 1 meter or even up to 2 meters. For large scale or commercial production, the column bed height is generally at least about 10 cm, 15 cm, or 20 cm, and up to about 25 cm or 30 cm.

The term “buffer” or “buffered solution” refers to a solution that is able to resist changes in pH. Often a buffer is made of a weak conjugate acid-base pair, for example, a weak acid and its conjugate base or a weak base and its conjugate acid. In some buffers, the buffering agent is supplied as a crystalline acid or base; for example, Tris is supplied as a crystalline base, which is dissolved in water to form a buffering solution. The pH of the buffering solution can be adjusted using an appropriate acid or base. For example, hydrochloric acid (HCl) can be used to adjust the pH of a Tris buffering solution. Other buffers are prepared by mixing two components, such as a free acid or base and a corresponding salt, in ratios that achieve the desired pH. For example, a sodium citrate buffer solution can be made and adjusted to the desired pH by combining citric acid and trisodium citrate to form a solution with the desired pH. Other buffers are made by mixing a buffer component and its conjugate acid or base. For example, a phosphate buffer can be made by mixing monobasic and dibasic sodium phosphate solutions in a ratio to achieve a desired pH. In another embodiment, a sodium bicarbonate buffer system can be prepared by combining solutions of sodium carbonate and sodium bicarbonate to form a buffer solution having a desired pH.

In some embodiments, the affinity chromatography column is equilibrated with an “equilibrating buffer” prior to loading. The terms “equilibrating buffer” and “equilibration buffer” refer interchangeably to a buffer that can be used to remove undesired residuals from the column matrix and to prepare the solid phase of the column matrix for loading the antibody, for example, by adjusting the pH of the column. For antibody purification, the pH of the equilibrating buffer is 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, the equilibration buffer includes a buffering agent such as tris(hydroxymethyl)aminomethane (often referred to as “Tris”) (pH range 6.0-8.0), 4-(2-hydroxyethyl)-1-piperazineethanesulfonic acid (HEPES) (pH range 6.8-8.2), 3-(N-morpholino)propanesulfonic acid (MOPS) (pH range 6.5-7.9), or other phosphate buffering agents (pH 5.8-8.0) at a concentration of at least about 10 mM, 25 mM, 50 mM, or 75 mM, and up to about 100 mM, 125 mM, or 150 mM. In some embodiments, the equilibration buffer includes up to about 100 mM Tris at a pH of 6.0 to 8.0. In some embodiments, the pH of the buffering solution can be adjusted using an appropriate acid or base, such as hydrochloric acid (HCl) or sodium hydroxide/potassium hydroxide (NaOH/KOH). In some embodiments, the equilibrating buffer comprises about 45 mM to about 55 mM Tris at a pH of 6.0 to 8.0. In some embodiments, the equilibrating buffer comprises 50 mM Tris at pH 7.4.

After equilibration, the affinity chromatography column is loaded with a solution comprising the antibody. In general, the concentration amount of antibody loaded onto the affinity chromatography column is at least about 10 mg/mL, 20 mg/mL, 30 mg/mL, 40 mg/mL, or 50 mg/mL, and up to about 55 mg/mL, 60 mg/mL, 70 mg/mL, 80 mg/mL, 90 mg/mL, or 100 mg/mL. In some embodiments, the amount of antibody loaded onto the affinity chromatography column is at least 25 mg/mL up to about 50 mg/mL. In some embodiments, the amount of antibody loaded onto the affinity chromatography column is about 50 mg/mL. The term “washing buffer” refers to a buffer that is passed over the column material after the antibody has been loaded onto the column. The washing buffer may serve to remove one or more contaminants, for example, host cell protein, from the column material, without substantial elution of the target. A wash step where the washing buffer is passed through the affinity chromatography column may be included during the affinity capture step. In some embodiments, the washing step occurs after the loading of the solution comprising the antibody onto the affinity chromatography column and before the affinity chromatography column is re-equilibrated.

The washing buffer has pH of 6.0 to 10.0. In some embodiments, the washing buffer has a pH of 7.0 to 9.0. In some embodiments, the process may include more than one wash buffer; for example, the process may include two different wash buffers. In some embodiments, the washing buffer comprises up to about 1 M arginine at a pH of 6.0 to 8.0. In some embodiments, the washing buffer comprises at least about 45 mM sodium caprylate at a pH of 6.0 to 10. In some embodiments, the washing buffer comprises at least about 45 mM sodium caprylate to about 4 M sodium chloride at a pH of 6.0 to 8.0. In some embodiments, the washing buffer comprises no more than 4 M sodium chloride at a pH of 6.0 to 8.0. In some embodiments, the washing buffer comprises about 58 mM sodium phosphate and about 0.5 M arginine at a pH of about 7.0. In some embodiments, the washing buffer comprises about 100 mM Tris, about 50 mM sodium caprylate, and about 2.5 M sodium chloride at pH 9.0. In some embodiments, the washing buffer comprises about 52 mM Tris and about 1.0 M sodium chloride at pH 7.4.

In particular embodiments, the affinity chromatography column is re-equilibrated with the equilibrating buffer after the antibody has been loaded onto the affinity column and prior to elution of the antibody. In particular embodiments, the equilibrating buffer comprises about 45 mM to about 55 mM at a pH 6.0 to 8.0.

The methods disclosed herein include eluting the antibody from the affinity chromatography column using an elution buffer to produce an eluate. The term “elution buffer” refers to a buffer used to elute (i.e., remove) the antibody from the column. The elution pH can vary depending upon the binding affinity of the antibody to the column. Some antibodies demonstrate a higher binding affinity and may require a lower elution pH. In general, the pH of the elution buffer is lower than the pH of the loading buffer. Typically, the elution buffer has a pH of 3.0, 3.1, 3.2, 3.3, 3.4, 3.5, 3.6, 3.7, 3.8, 3.9, or 4.0. Examples of elution buffers include buffers including sodium citrate, citric acid, or acetic acid at a concentration of at least about 25 mM, 50 mM and up to about 100 mM, 150 mM, or 200 mM. In some embodiments, the elution buffer comprises at least about 25 mM, 50 mM, and up to about 60 mM sodium citrate at pH of 3.0, 3.1, 3.2, 3.3, 3.4, 3.5, 3.6, 3.7, 3.8, 3.9, or 4.0. In some embodiments, the elution buffer comprises about 45 mM to about 55 mM sodium citrate at a pH of 3.0 to 4.0. In some embodiments, the elution buffer comprises about 50 mM sodium acetate at pH 3.6.

The term “eluate” refers to a solution that is obtained by elution of an adsorbed material bound to the solid phase in affinity chromatography. For example, in a bind and elute chromatography process, the eluate is the solution that comprises the antibody after the unwanted proteins have passed through the column.

The eluate can be monitored using techniques well known to those skilled in the art, for example by monitoring the absorbance using a spectrophotometer.

In some embodiments, the affinity chromatography step has a recovery of at least about 80%, 85%, 90%, 91%, 92%, 93%, 94%, or 95%, and up to about 96%, 97%, 98%, 99%, or 100%. In some embodiments, recovery is at least about 99% to 100% following the affinity chromatography step. Recovery can be determined, for example, by calculating the percentage of protein in the eluate relative to the amount that was loaded onto the column.

In some embodiments, less than about 5%, 4%, 3%, 2%, or 1% of the antibody forms an aggregate following the affinity chromatography step as determined by high pressure size exclusion chromatography (HP-SPEC). In some embodiments, less than about 1% of the antibody forms an aggregate following the affinity chromatography step. In some embodiments, at least about 95%, 96%, 97%, 98%, or 99% of the target antibody is present as a monomer following the affinity chromatography step as measured by HP-SEC. In some embodiments, at least about 98% of the antibody is present as a monomer following the affinity chromatography step. In some embodiments, host cell protein is present at less than about 200 ng/mg, 190 ng/mg, 175 ng/mg, 160 ng/mg, 150 ng/mg, or 140 ng/mg following the affinity chromatography step as measured by methods known in the art, such as ELISA. In some embodiments, host cell protein is present at less than about 175 ng/mg following the affinity chromatography step. In some embodiments, DNA content is present at less than about 110 pg/mg, 100 pg/mg, 90 pg/mg, 80 pg/mg, 70 pg/mg, 60 pg/mg, 50 pg/mg, 40 pg/mg, or 30 pg/mg following the affinity chromatography step as measured by methods known to one having skill in the art, such as real-time PCR (qPCR). In some embodiments, DNA content is less than about 95 pg/mg following the affinity chromatography step.

### 3. Low pH Treatment

The purification methods disclosed herein include low pH treatment of the eluate from the affinity chromatography by using acidic titrant and/or basic titrant solutions that reduce or increase the pH of the eluate. In particular embodiments, the low pH is a pH between 3 and 5. In particular embodiments, the solution is a virus inactivation solution capable of effectively inactivating enveloped viruses. In some embodiments, the low pH viral inactivation includes titrating the eluate to a low pH between 3 and 5 using an acidic virus inactivation titrant solution, incubating the eluate and acidic titrant solution in the titrated pH range, and neutralizing the eluate and acidic titrant solution to neutral pH using a basic titrant solution. The choice of pH level depends on the stability profile of the antibody and other buffer components. In some embodiments, the acidic titrant solution comprises at least about 100 mM glycine to about 500 mM glycine at pH of 2.0 to pH 3.0. In some embodiments, the acidic titrant solution comprises about 248 mM glycine at pH of 2.35. Typically, the titrated solution is incubated for at least about 15, 30, or 45 minutes, and up to about 1, 2, 3, 4, or 5 hours. After viral inactivation, the pH of the antibody solution can be adjusted to a more neutral pH, for example, between 4.5 to 8.5, or between 7.2 and 7.6 prior to continuing the purification process. In some embodiments, the basic titrant solution used to adjust the antibody solution to a more neutral pH comprises about 0.5 M Tris to about 1.0 M Tris. In some embodiments, the basic titrant solution used to adjust the antibody solution to a more neutral pH comprises 0.5 M Tris.

In some embodiments, the low pH viral inactivation step has a recovery of at least about 80%, 85%, 90%, 91%, 92%, 93%, 94%, or 95%, and up to about 96%, 97%, 98%, 99%, or 100%. In some embodiments, recovery is at least about 95% following the low pH viral inactivation step. Recovery can be determined, for example, by calculating the percentage of antibody in the eluate relative to the amount that was loaded onto the column. In some embodiments, less than about 5%, 4%, 3%, 2%, or 1% of the antibody forms an aggregate following the low pH viral inactivation step as determined by high pressure size exclusion chromatography (HP-SPEC). In some embodiments, less than about 2% of the antibody forms an aggregate following the low pH viral inactivation step. In some embodiments, at least about 95%, 96%, 97%, 98%, or 99% of the antibody is present as a monomer following the low pH viral inactivation step as measured by HP-SEC. In some embodiments, at least about 98% of the target antibody is present as a monomer following the low pH viral inactivation step. In some embodiments, the host cell protein is present at less than about 100 ng/mg, 95 ng/mg, 90 ng/mg, 85 ng/mg, or 80 ng/ mg following the low pH viral inactivation step. In some embodiments, host cell protein is present at less than about 90 ng/mg following the low pH viral inactivation step. In some embodiments, DNA content is less than about 1 pg/mg, 0.9 pg/mg, 0.8 pg/mg, 0.7 pg/mg, 0.6 pg/mg, 0.5 pg/mg, 0.4 pg/mg, 0.3 pg/mg, 0.2 pg/mg, or 0.1 pg/mg following the low pH viral inactivation step as measured by methods known to one having skill in the art, such as real-time PCR (qPCR). In some embodiments, DNA content is less than about 0.5 pg/mg following the low pH viral inactivation step.

### 4. Polishing Steps

The one or more polishing chromatography steps occur after the capture step and provide additional viral, host cell protein (HCP), endotoxin, and/or DNA clearance, and also assist in the removal of aggregates, unwanted product variants, and other minor contaminants. Polishing steps generally include one or more chromatographic steps such as ion exchange chromatography, mixed mode chromatography, hydrophobic interaction chromatography, and combinations thereof.

In some embodiments, the purification method includes at least one ion exchange chromatography step. The term “ion exchange chromatography” refers to a chromatographic process using an immobile matrix that carries covalently bound charged substituents. The “ion exchange material” has the ability to exchange its counter ions, which are not covalently bound, for similarly charged binding partners or ions in the surrounding solution. Polypeptides have numerous functional groups that can have either positive or negative charges. Ion exchange chromatography separates polypeptides based on net charge, which is dependent on the pH and/or ionic concentration of the mobile phase. Polypeptides can thus be separated by adjusting the pH and/or ionic concentration of the mobile phase. In some embodiments, the antibody is captured by the ion exchange chromatography column and then eluted (also referred to as “bind and elute” mode). In some embodiments, the antibody flows through the ion exchange chromatography column and contaminants are bound (also referred to as a “flow through mode”). Elution from an ion exchange material is generally achieved by increasing the ionic strength of the buffer to compete with the antibody for charged sites of the ion exchange matrix. The elution process can be gradual (gradient elution) or stepwise (step elution) and the eluate can be monitored using a UV spectrophotometer set at OD₂₈₀ nm.

Depending on the charge of the counter ions, “ion exchange chromatography” can be referred to as “cation exchange,” “anion exchange,” or “mixed-mode ion exchange.”

The term “anion exchange” refers to a chromatographic method having a solid phase that is positively charged with free anions available for exchange with anions in an aqueous solution passed over or through the solid phase. The anion exchange columns are typically operated in a flow through mode, such that negatively charged impurities are bound to the resin while the positively charged target polypeptide is recovered in the flow-through stream. However, anion exchange columns may also be used in a bind and elute mode, depending upon the pH of the antibody and the impurities to be removed. Examples of positively charged groups that are used in anion exchange include weakly basic groups such as diethylamino ethyl (DEAE) or dimethylamino ethyl (DMAE) and strongly basic groups such as quaternary amine (Q) groups, trimethylammonium ethyl (TMAE), or quaternary aminoethyl (QAE).

The term “cation exchange” refers to a chromatographic method having a solid phase that is negatively charged with free cations available for exchange with cations in an aqueous solution passed over or through the cation exchange chromatography column. Cation exchange chromatography can be used to purify an antibody if the antibody is maintained under conditions in which the antibody is positively charged. For example, the solution can be titrated so that the solution pH is lower than the isoelectric point of the antibody. Other positively charged impurities may also be bound to the cation column resin in addition to the target antibody. As such, the antibody can be recovered by elution from the column under conditions (e.g., pH and salt concentration) in which the antibody elutes while impurities remain bound to the resin. Cation exchange resins can include strong acidic ligands such as sulphopropyl, sulfoethyl, and sulfoisobutyl groups, or weak acidic ligands such as carboxyl groups. Examples of commonly used cation exchange resins include carboxymethyl (CM), sulfoethyl (SE), sulfopropyl (SP), phosphate (P), and sulfonate (S) resins.

In some embodiments, the eluate obtained from the low pH treatment is subjected to one, two, or more than two ion exchange separation steps in which the second ion exchange separation involves a separation based on the opposite charge than the first ion exchange separation. For example, if an anion exchange step is employed after capture, the second ion exchange chromatography step may be cation exchange chromatography and vice versa. Alternatively, in some embodiments, the purification process may include only an anion exchange step or only a cation exchange step.

In some embodiments, the neutralized elute from the low pH treatment is subjected to anion exchange chromatography and the flow-through is collected. The collected flow-through is then subjected to a cation exchange chromatography and the antibody is eluted from the cation exchange chromatography to produce a cation exchange chromatography product comprising the antibody.

### 5. Anion Exchange

In some embodiments, the anion exchange chromatography comprises resin-based chromatography or membrane chromatography. Resin-based anion exchange can be facilitated by resins such as Capto Q resin, Tosho Super Q, and POROS HQ. Membrane chromatography can be facilitated by, for example, Sartobind Q®, Mustang Q®, and ChromaSorb®.

In some embodiments of the anion exchange chromatography polishing step, the anion exchange chromatography apparatus is equilibrated with an equilibrating buffer prior to being loaded with eluate product from the low pH treatment step. The equilibrating buffer can be the same as the affinity equilibration buffer. Anion exchange equilibration buffers are used to remove undesired residual from the column matrix and to prepare the solid phase of the column matrix for loading, for example, by adjusting the pH of the column. The pH of the equilibration buffer is 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, the equilibration buffer comprises about 100 mM Tris at a pH of 6.0 to 8.0. In some embodiments, the pH of the buffering solution can be adjusted using an appropriate acid or base, such as hydrochloric acid (HCl) or sodium hydroxide/potassium hydroxide (NaOH/KOH). In some embodiments, the equilibrating buffer comprises at least about 45 mM up to about 55 mM Tris at a pH 7.0 to pH 8.0. In some embodiments, the equilibrating buffer comprises at about 50 mM Tris at pH 7.4.

After the anion exchange column is equilibrated, the neutralized eluate comprising the target antibody is loaded onto the anion exchange chromatography apparatus. In some embodiments, at least about 50 mg/mL, 75 mg/mL, 100 mg/mL, 150 mg/mL, 400 mg/mL, and up to about 450 mg/mL, or 500 mg/mL of the antibody is loaded onto the anion exchange chromatography column. In some embodiments, up to about 500 mg/mL of target antibody is subjected to anion exchange chromatography. In some embodiments, up to about 150 mg/mL target antibody is loaded onto the anion exchange chromatography column. In some embodiments, up to about 75 mg/mL antibody is loaded onto the anion exchange chromatography column.

The target antibody is captured in the neutralized eluate that flows through the anion exchange chromatography apparatus. In some embodiments, the neutralized eluate is chased with a chase solution comprising 45 mM to 55 mM Tris at a pH of 3.0 to 4.0. The chase solution that flows through the anion exchange column is captured to produce a flow-through collection comprising the target antibody.

In some embodiments, the anion exchange chromatography step has a recovery of at least about 80%, 85%, 90%, 91%, 92%, 93%, 94%, or 95%, and up to about 96%, 97%, 98%, 99%, or 100%. In some embodiments, recovery is at least about 95% following the anion exchange chromatography step. Recovery can be determined, for example, by calculating the percentage of antibody in the eluate relative to the amount that was loaded onto the column. In some embodiments, less than about 5%, 4%, 3%, 2%, or 1% of the antibody forms an aggregate following the anion exchange chromatography step as determined by high pressure size exclusion chromatography (HP-SPEC). In some embodiments, less than about 2% of the antibody forms an aggregate following the anion exchange chromatography step. In some embodiments, at least about 95%, 96%, 97%, 98%, or 99% of the target antibody is present as a monomer following the anion exchange chromatography step as measured by HP-SEC. In some embodiments, at least about 98% of the target antibody is present as a monomer following the anion exchange chromatography step. In some embodiments, host cell protein is present at less than about 15 ng/mg, 14 ng/mg, 13 ng/mg, 12 ng/mg, 11 ng/mg, 10 ng/mg, 9 ng/mg, 8 ng/mg, 7 ng/mg, 6 ng/mg, 5 ng/mg, 4 ng/mg, 3 ng/mg, 2 ng/mg, or 1 ng/mg following the anion exchange chromatography step as measured by methods known in the art, such as ELISA. In some embodiments, host cell protein is present at less than about 12 ng/mg following the anion exchange chromatography step. In some embodiments, DNA content is present at less than about 1 pg/mg, 0.9 pg/mg, 0.8 pg/mg, 0.7 pg/mg, 0.6 pg/mg, 0.5 pg/mg, 0.4 pg/mg, 0.3 pg/mg, 0.2 pg/mg, or 0.1 pg/mg following the anion exchange chromatography step as measured by methods known to one having skill in the art such as real-time PCR (qPCR). In some embodiments, DNA content is less than about 0.5 pg/mg following the anion exchange chromatography step.

In some embodiments, an optional step of pre-equilibrating the anion exchange chromatography apparatus with a pre-equilibration buffer is performed before equilibration. In some embodiments, the pre-equilibration buffer comprises at least about 50, or 55 mM, up to about 60, 70, 80, 90, or 100 mM Tris, and at least about 0.15, 0.2, 0.3, 0.4, 0.5, 0.6, or 0.75 M, up to about 0.8, 0.9, 1.0, 1.25, 1.5 M sodium chloride at a pH of 5.0 to 10.0. In some embodiments, the pre-equilibration buffer comprises at least about 50 mM up to about 60 mM Tris, and at least about 0.5 M up to about 1.0 M sodium chloride at a pH of 7.0 to 8.0. In some embodiments, the pre-equilibration buffer comprises about 52 mM Tris and about 1.0 M sodium chloride at pH 7.4.

### 6. Cation Exchange

Once the flow-through collection from the anion exchange chromatography step is collected, the flow-through collection is subjected to cation exchange chromatography. In some embodiments, the cation exchange chromatography is facilitated by a resin-based column, including, but not limited to, for example, POROS HS50 (Applied Biosystems), Nuvia HR-S(Bio-Rad), Toyopearl AF-Epoxy-650 (Tosoh Bioscience), and SP Sepharose Fast Flow (GE Healthcare). In some embodiments, the cation exchange chromatography is facilitated by POROS Strong Cation Exchange Media 50 micron column.

In some embodiments of the cation exchange chromatography polishing step, the cation exchange chromatography column is equilibrated with cationic exchange equilibrium buffer prior to being loaded with the flow-through product from the anion exchange polishing step. The pH of the cationic exchange equilibrium buffer is 4.5 to 5.5. In some embodiments, the cationic exchange equilibrium buffer comprises at least 40 mM up to 60 mM sodium acetate at a pH of 4.5 to 5.5. In some embodiments, the cationic exchange equilibrium buffer comprises 50 mM sodium acetate at pH 5.0.

In some embodiments, the cation exchange chromatography column is loaded with up to about 100 mg/mL of the flow-through collection comprising the target antibody, washed with a cation exchange washing buffer, and eluted from the cation exchange chromatography column using a cation exchange elution buffer.

In some embodiments, the cation exchange washing buffer comprises at least about 15 mM up to about 50 mM Histidine/Histidine-HCl at a pH of 5.0 to 7.0. In some embodiments, the cation exchange washing buffer comprises at least about 20 mM up to about 30 mM Histidine/Histidine-HCl at a pH of 5.0 to 6.0. In some embodiments the cation exchange washing buffer comprises 26 mM Histidine/Histidine-HCl at pH 5.8.

The cation exchange elution buffer comprises at least 10 mM up to 30 mM Histidine/Histidine-HCl, and about 115 mM to about 200 mM sodium chloride at a pH of 5.0 to 7.0. In some embodiments, the cation exchange elution buffer comprises 20 mM Histidine/ Histidine-HCl, 500 mM NaCl, pH 6.0. In some embodiments, the cation exchange eluting buffer comprises about 20 mM Histidine/Histidine-HCl and about 115 mM sodium chloride at pH 6.0.

In some embodiments, the cation exchange chromatography step has a recovery of at least about 75%, 80%, 85%, or 90%, and up to about 93%, 96%, 97%, 98%, or 99%. In some embodiments, recovery is at least about 88% or 89% up to about 91% or 92% following the cation exchange chromatography step. Recovery can be determined, for example, by calculating the percentage of antibody in the eluate relative to the amount that was loaded onto the column. In some embodiments, less than about 5%, 4%, 3%, 2%, or 1% of the antibody forms an aggregate following the cation exchange chromatography step as determined by HP-SPEC. In some embodiments, less than about 1% of the antibody forms an aggregate following the cation exchange chromatography step as determined by HP-SPEC. In some embodiments, at least about 95%, 96%, 97%, 98%, or 99% of the target antibody is present as a monomer following the cation exchange chromatography step as measured by HP-SEC. In some embodiments, at least about 99% of the target antibody is present as a monomer following the cation exchange chromatography step as determined by HP-SPEC. In some embodiments, the host cell protein is present at less than about 15 ng/mg, 14 ng/mg, 13 ng/mg, 12 ng/mg, 11 ng/mg, 10 ng/mg, 9 ng/mg, 8 ng/mg, 7 ng/mg, 6 ng/mg, 5 ng/mg, 4 ng/mg, 3 ng/mg, 2 ng/mg, or 1 ng/mg following the cation exchange chromatography step as measured by methods known to one having skill in the art, such as ELISA. In some embodiments, host cell protein is present at less than about 5 ng/mg following the cation exchange chromatography step. In some embodiments, DNA content is present at less than about 1 pg/mg, 0.9 pg/mg, 0.8 pg/mg, 0.7 pg/mg, 0.6 pg/mg, 0.5 pg/mg, 0.4 pg/mg, 0.3 pg/mg, 0.2 pg/mg, or 0.1 pg/mg following the cation exchange chromatography step as measured by methods known to one having skill in the art, such as real-time PCR (qPCR). In some embodiments, DNA content is less than about 0.2 pg/mg following the cation exchange chromatography step.

In some embodiments, the elution of the cation exchange chromatography step is supplemented with at least one organic modifier. The organic modifier induces a favorable and changed selectivity between the unwanted related impurity or impurities and the antibody and the ion exchanger. Suitable organic modifiers include, but are not limited to, arginine, urea, glycine, C₁-₆-alkanol, C₁-₆-alkenol, or C₁-₆-alkynol, guanidine HCl, or C₁-₆-alkanoic acid, such as acetic acid, C₂-₆-glycol such as propylene glycol, and C₃-₇-polyalcohol, including sugars or mixtures thereof.

### 7. Further Purification

In some embodiments, a viral clearance step such as viral filtration (nanofiltration), is included in the purification scheme following the cation exchange chromatography step. The viral clearance step is performed to remove small non-enveloped viruses which are more resistant to the viral inactivation treatment. Virus-retentive filters are commercially available and include ultrafilters or microfilters such as hydrophilic polyether sulfone (PES), hydrophilic polyvinylidene (PVDF), and regenerated cellulose filters. Based on the size of viruses that are removed, virus filters can be categorized into retrovirus filters and parvovirus filters. Examples of virus filters include, but are not limited to, vPro+ (Millipore), Planova 20N (Asahi Kasei), and Planova BioEX (Asahi Kasei).

In some embodiments, the nanofiltration step has a recovery of at least about 75%, 80%, 85%, or 90%, and up to about 93%, 96%, 97%, 98%, 99%, or 100%. In some embodiments, recovery is at least about 98% to about 99% following the nanofiltration step. Recovery can be determined, for example, by calculating the percentage of antibody in the eluate relative to the amount that was loaded onto the column. In some embodiments, less than about 5%, 4%, 3%, 2%, or 1% of the antibody forms an aggregate following the nanofiltration step as determined by HP-SPEC. In some embodiments, less than about 1% of the antibody forms an aggregate following the nanofiltration chromatography step as determined by HP-SPEC. In some embodiments, at least about 95%, 96%, 97%, 98%, or 99% of the target antibody is present as a monomer following the nanofiltration step as measured by HP-SEC. In some embodiments, at least about 99% of the target antibody is present as a monomer following the nanofiltration step as determined by HP-SPEC.

In some embodiments, the purification scheme includes an ultrafiltration (UF) and/or diafiltration (DF) step to further purify and concentrate the antibody sample following nanofiltration. UF/DF can increase the concentration of the target polypeptide as well as replace buffering salts with a particular formulation buffer. Ultrafiltration (UF) refers to a type of membrane filtration in which hydrostatic pressure forces a liquid against a semipermeable membrane. In some embodiments, UF is performed with Tangential Flow Filtration (TFF), including virus filtration and high performance tangential flow filtration (HPTFF). TFF membranes have nominal molecular weight limits in the range of 1 kD up to 1000 kD, where virus filtration membrane NMWL ratings range from 100 kD to 500 kD, or up to 0.05 µm and HPTFF Membrane NMWLs used for HPTFF are in the range of 10 kD to 300 kD. In some embodiments, the TFF membranes include Omega-T (Pall), P3 Ultracell RC, C screen (Millipore), P3 Ultracell RC, D screen (Millipore), Hydrosart (Sartorius), and Hydrosart ECO (Sartorius). Suspended solids and solutes of high molecular weight, such as the target polypeptide, are retained in the retentate, while water and low molecular weight solutes pass through the membrane in the filtrate. In this manner, the target antibodies are concentrated whereas liquid and salt are removed. Generally, the low molecular weight composition in the concentrate remains constant so the ionic strength of the concentrated solution remains relatively constant. “Diafiltration” refers to a method that uses ultrafiltration membranes to remove, replace, or lower the concentrations of salts or buffering components from solutions containing proteins, such as antibodies, peptides, nucleic acids, and other biomolecules. Continuous diafiltration (also referred to as constant volume diafiltration) involves washing out the original buffer salts (or other low molecular weight species) in the retentate by adding water or a new buffer, such as a formulation buffer, to the retentate to form a formulation containing the recombinantly produced polypeptide. Typically, the new buffer is added at the same rate as filtrate is being generated such that the retentate volume and product concentration does not change appreciably during diafiltration.

In some embodiments, the ultrafiltration/diafiltration step has a recovery of at least about 85%, 90%, 93%, 96%, 97%, 98%, 99%, or 100%. In some embodiments, recovery is at least 99% following the ultrafiltration/diafiltration step. Recovery can be determined, for example, by calculating the percentage of antibody in the eluate relative to the amount that was loaded onto the column. In some embodiments, less than about 5%, 4%, 3%, 2%, or 1% of the antibody forms an aggregate following the ultrafiltration/diafiltration step as determined by HP-SPEC. In some embodiments, less than about 1% of the antibody forms an aggregate following the ultrafiltration/diafiltration step as determined by HP-SPEC. In some embodiments, at least about 95%, 96%, 97%, 98%, or 99% of the target antibody is present as a monomer following the ultrafiltration/diafiltration step as measured by HP-SEC. In some embodiments, at least about 99% of the target antibody is present as a monomer following the ultrafiltration/diafiltration step as determined by HP-SPEC. In some embodiments, host cell protein is present at less than about 15 ng/mg, 14 ng/mg, 13 ng/mg, 12 ng/mg, 11 ng/mg, 10 ng/mg, 9 ng/mg, 8 ng/mg, 7 ng/mg, 6 ng/mg, 5 ng/mg, 4 ng/mg, 3 ng/mg, 2 ng/mg, or 1 ng/mg following the ultrafiltration/diafiltration step as measured by methods known in the art, such as ELISA. In some embodiments, host cell protein is present at less than about 1.5 ng/mg following the ultrafiltration/diafiltration step as measured by methods known in the art, such as ELISA. In some embodiments, DNA content is present at less than about 0.5 pg/mg, 0.4 pg/mg, 0.3 pg/mg, 0.2 pg/mg, 0.1 pg/mg, 0.09 pg/mg, 0.08 pg/mg, 0.07 pg/mg, 0.06 pg/mg, 0.05 pg/mg, 0.04 pg/mg, 0.03 pg/mg, 0.02 pg/mg, or 0.01 pg/mg following the ultrafiltration/diafiltration step as measured by methods known to one having skill in the art, such as real-time PCR (qPCR). In some embodiments, DNA content is less than about 0.05 pg/mg following the ultrafiltration/diafiltration step.

In some embodiments, the antibody purification process has a recovery of at least about 50%, 55%, or 60%, and up to about 65%, 70%, 75%, 80%, 85%, 90%, or 95%. In some embodiments, the purification recovery is at least about 60% to about 70%. In some embodiments, at least about 95%, 96%, 97%, 98%, or 99% of the target antibody is present as a monomer following the target antibody purification process as measured by HP-SEC. In some embodiments, at least about 99% of the target antibody is present as a monomer following the target antibody purification process. In some embodiments, less than about 5%, 4%, 3%, 2%, or 1% of the antibody forms an aggregate following the target antibody purification process as determined by HP-SPEC. In some embodiments, less than about 1% of the antibody forms an aggregate following the antibody purification process. In some embodiments, DNA content is present at less than about 1 pg/mg, 0.9 pg/mg, 0.8 pg/mg, 0.7 pg/mg, 0.6 pg/mg, 0.5 pg/mg, 0.4 pg/mg, 0.3 pg/mg, 0.2 pg/mg, or 0.1 pg/mg following the target antibody purification process as measured by methods known to one having skill in the art, such as real-time PCR (qPCR). In some embodiments, DNA content is less than about 0.2 pg/mg following the target antibody purification process. In some embodiments, host cell protein is present at less than about 15 ng/mg, 14 ng/mg, 13 ng/mg, 12 ng/mg, 11 ng/mg, 10 ng/mg, 9 ng/mg, 8 ng/mg, 7 ng/mg, 6 ng/mg, 5 ng/mg, 4 ng/mg, 3 ng/mg, 2 ng/mg, or 1 ng/mg following the target antibody purification process as measured by methods known in the art, such as ELISA. In some embodiments, host cell protein is present at less than about 10 ng/mg following the target antibody purification process.

The terms “MEDI4736” and “durvalumab,” as used herein, refer to an antibody that selectively binds human anti-PD-L1 and blocks the binding of PD-L1 to PD-1 and CD80 receptors, as disclosed in U.S. Pat. Nos. 8,779,108 and 9,493,565, which are each incorporated by reference herein in their entireties. The fragment crystallizable (Fc) domain of durvalumab contains a triple mutation in the constant domain of the IgG 1 heavy chain that reduces binding to the complement component C1q and the Fcy receptors responsible for mediating antibody-dependent cell-mediated cytotoxicity (ADCC). Durvalumab can relieve PD-L1-mediated suppression of human T-cell activation in vitro and inhibits tumor growth in a xenograft model via a T-cell dependent mechanism.

In some embodiments, the purification processes disclosed herein can be used to purify an anti-PD-L1 monoclonal antibody. In some embodiments, the anti-PD-L1 monoclonal antibody is durvalumab. In some embodiments, the light chain variable domain of the purified anti-PD-L1 monoclonal antibody comprises the amino acid sequence of SEQ ID NO: 1 and the heavy chain variable domain comprises the amino acid sequence of SEQ ID NO: 2. In some embodiments, the anti-PD-L1 antibody comprises a heavy chain variable domain comprising a CDR1-H of the amino acid sequence of SEQ ID NO: 3, a CDR2-H of the amino acid sequence of SEQ ID NO: 4, and a CDR3-H of the amino acid sequence of SEQ ID NO: 5. In some embodiments, the anti-PD-L1 antibody comprises a light chain variable domain comprising a CDR1-L of the amino acid sequence of SEQ ID NO: 6, a CDR2-L of the amino acid sequence of SEQ ID NO: 7, and a CDR3-L of the amino acid sequence of SEQ ID NO: 8. These sequences are shown in Table 1.

TABLE 1

| SEQ ID NO: | Amino Acid Sequence | Human Anti-PD-L1 mAb durvalumab |
|---|---|---|
| 1 | EIVLTQSPGTLSLSPGERATLSCRASQRVSSSYLAWYQQKPGQA PRLLIYDASSRATGIPDRFSGSGSGTDFTLTISRLEPEDFAVYYC QQYGSLPWTFGQGTKVEIK | Light Chain (VL) |
| 2 | EVQLVESGGGLVQPGGSLRLSCAASGFTFSRYWMSWVRQAPG KGLEWVANIKQDGSEKYYVDSVKGRFTISRDNAKNSLYLQMN SLRAEDTAVYYCAREGGWFGELAFDYWGQGTLVTVSS | Heavy Chain (VH) |
| 3 | GFTFSRYWMS | CDR1-H |
| 4 | NIKQDGSEKYYVDSVKG | CDR2-H |
| 5 | EGGWFGELAFDY | CDR3-H |
| 6 | RASQRVSSSYLA | CDR1-L |
| 7 | DASSRAT | CDR2-L |
| 8 | QQYGSLPWT | CDR3-L |

### 8. Re-Use of Chromatography Material

The expense of affinity chromatography materials makes the ability to re-use such materials a priority. Additionally, the re-use of the anion exchange chromatography and cation exchange chromatography materials is desired. While these materials are available for re-use, their re-use is not necessary to the disclosed purification process.

Under the current disclosure, the affinity chromatography column may be re-used. In some embodiments, the affinity chromatography column is stripped (cleaned of excess or leftover debris) for re-use with a stripping buffer. In some embodiments, the stripping buffer comprises about 45 mM to about 55 mM glycine at a pH of 2.0 to 3.0. In some embodiments, the stripping buffer comprises about 50 mM glycine at pH 2.3. In some embodiments, the affinity chromatography column is sanitized with a sanitizing buffer. In general, sanitizing buffers comprise at least about 0.1 N NaOH up to about 1.5 N NaOH. In some embodiments, the sanitizing buffer comprises about 0.1 N NaOH. In some embodiments, the sanitizing buffer comprises about 1 N NaOH. In some embodiments, the re-equilibrating buffer comprises about 45 mM to about 55 mM Tris at a pH of 7.0 to 8.0. In some embodiments, the re-equilibrating buffer comprises about 50 mM Tris at pH 7.4. In some embodiments, the affinity chromatography column is stored in a storage buffer comprising about 90 mM to about 100 mM sodium acetate and about 1% to about 2% benzyl alcohol at a pH of 5.0 to 6.0. In some embodiments, the storage buffer comprises about 100 mM sodium acetate and about 2% benzyl alcohol at pH 5.0.

In some embodiments, the anion exchange chromatography column is stripped for re-use with a stripping buffer. In some embodiments, the stripping buffer comprises at least about 40 mM, 45 mM, or 50 mM, up to about 55 mM, 60 mM, or 65 mM Tris. In some embodiments, the stripping buffer comprises at least about 0.8 N, 0.9 N, 1.0 N, 1.1 N, or 1.2 N NaOH. In some embodiments the stripping buffer is in the pH range of 5.0 to 9.0. In some embodiments, the stripping buffer comprises 52 mM Tris, 1 M NaCl, pH 7.4. In some embodiments, the anion exchange chromatography column is sanitized with a sanitizing buffer. In general, sanitizing buffers comprise at least about 0.1 N NaOH up to about 1.5 N NaOH. In some embodiments, the sanitizing buffer comprises about 0.1 N NaOH. In some embodiments, the sanitizing buffer comprises about 1 N NaOH. In some embodiments, the anion exchange chromatography column is stored in a storage buffer comprising about 0.1 N NaOH.

The cation exchange chromatography column is also reusable when stripped, sanitized, and stored. In some embodiments, the stripping buffer comprises at least about 40 mM, 45 mM, or 50 mM, up to about 55 mM, 60 mM, or 65 mM Tris. In some embodiments, the stripping buffer comprises at least about 0.8 N, 0.9 N, 1.0 N, 1.1 N, or 1.2 N NaOH. In some embodiments the stripping buffer is in the pH range of 5.0 to 9.0. In some embodiments, the cation exchange stripping buffer comprises 52 mM Tris, 1 M NaCl, pH 5.4. In general, sanitizing buffers comprise at least about 0.1 N NaOH up to about 1.5 N NaOH. In some embodiments, the sanitizing buffer comprises about 0.1 N NaOH. In some embodiments, the sanitizing buffer comprises about 1 N NaOH. In some embodiments, the anion exchange chromatography column is stored in a storage buffer comprising about 0.1 N NaOH.

In particular embodiments, a purified antibody composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; and wherein a main form of the antibody comprises greater than, or equal to, 45% of the protein in the composition as measured using capillary isoelectric focusing (cIEF) of the composition. In other embodiments, a purified antibody composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; and wherein acidic forms of the antibody comprise 45% to 50% of the protein in the composition as measured using cIEF of the composition. In other embodiments, a purified antibody composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; and wherein a basic form of the antibody comprises 18% to 23% of the protein in the composition as measured using cIEF of the composition. In other embodiments, a purified antibody composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; wherein acidic forms of the antibody comprise 45% to 50% of the protein in the composition as measured using cIEF of the composition; and wherein a basic form of the antibody comprises 18% to 23% of the protein in the composition as measured using cIEF of the composition.

In particular embodiments, a purified antibody composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein 1.5% - 2.5% of the anti-PD-L1 antibody forms an aggregate as determined by high-pressure size exclusion chromatography (HP-SPEC); and wherein 97% - 98% of the anti-PD-L1 antibody is present as a monomer as measured by HP-SEC.

In particular embodiments, a purified antibody preparation disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; and wherein the glycan structures of the anti-PD-L1 antibody comprise GOf, G1f, G2f, and G0 glycoforms. In some embodiments, the glycan structures of the anti-PD-L1 antibody have a content greater than about 90% for the G0f, G1f, G2f, and G0 forms. In some embodiments, the composition of the anti-PD-L1 antibody comprises about 65-75% G0fcontent, 13-23% G1f content, 0-3% content G2f, and 0-4% G0 content. In other embodiments, the composition of the anti-PD-L1 antibody comprises about 71.9% G0f content, 18.4% G1f content, 1.5% content G2f, and 1.9% G0 content.

### EXAMPLES

The Examples that follow are illustrative of specific embodiments of the disclosure, and various uses thereof. They are set forth for explanatory purposes only and should not be construed as limiting the scope of the disclosure in any way.

### Materials Used in Examples

Buffers used in the following Examples are shown in Table 2. The table indicates the steps for which the particular buffer can be used.

TABLE 2

| | |
|---|---|
| Step(s) | Buffer |
| Affinity capture Low pH virus inactivation Anion exchange chromatography | 50 mM Tris, pH 7.4 |
| | 52 mM Tris, 1 M NaCl, pH 7.4 |
| | 100 mM Tris, 50 mM sodium caprylate, 2.5 M NaCl, pH 9.0 |
| | 58 mM sodium phosphate, 0.5 M arginine, pH 7.0 |
| | 50 mM sodium acetate, pH 3.6 |
| | 248 mM Glycine, pH 2.35 |
| | 100 mM sodium acetate, 2% benzyl alcohol, pH 5.0 |
| | 500 mM Glycine, pH 2.35 |
| | 1 M Tris, pH 9.0 |
| | 0.5 M Tris |
| Sanitization | 1 N NaOH |
| | 0.1 NNaOH |
| Cation exchange chromatography | 50 mM sodium acetate, pH 5.0 |
| | 20 mM Histidine, 15 mM NaCl, pH 6.0 |
| | 50 mM sodium acetate, pH 5.0 |
| | 20 mM Histidine/ Histidine-HCl, 15 mM NaCl, pH 6.0 |
| | 26 mM Histidine/ Histidine-HCl, pH 5.8 |
| | 20 mM Histidine/ Histidine-HCl, 75 mM NaCl, pH 6.9 |
| | 20 mM Histidine/ Histidine-HCl, 115 mM NaCl, pH 6.0 |
| | 20 mM Histidine/ Histidine-HCl, 125 mM NaCl, pH 6.0 |
| | 20 mM Histidine/ Histidine-HCl, 130 mM NaCl, pH 6.0 |
| | 20 mM Histidine/ Histidine-HCl, 150 mM NaCl, pH 6.0 |
| | 50 mM Histidine, 2.0 M NaCl, pH 5.5 |

### Example 1. Development of Affinity Chromatography

Monoclonal antibodies were produced in a bioreactor and harvested through centrifugation as is known in the art. Briefly, antibodies were produced in either CHO or NS0 cell lines and ranged in IgG subclass of IgG1, IgG2, and IgG4.

In order to determine the optimized method of the capture step, cation exchange chromatography and anion exchange chromatography were evaluated as the first step in the capture process. The evaluation criteria included product quality, facility fit, throughput, number of differences between products, simplicity in development and operation, and ease of alignment when upscaling to the commercial purification platform. Protein A had a better overall evaluation than that of cation exchange chromatography. Protein A purification processes delivered high quality products while also providing significant advantages for HCP as well as aggregate and fragment clearance.

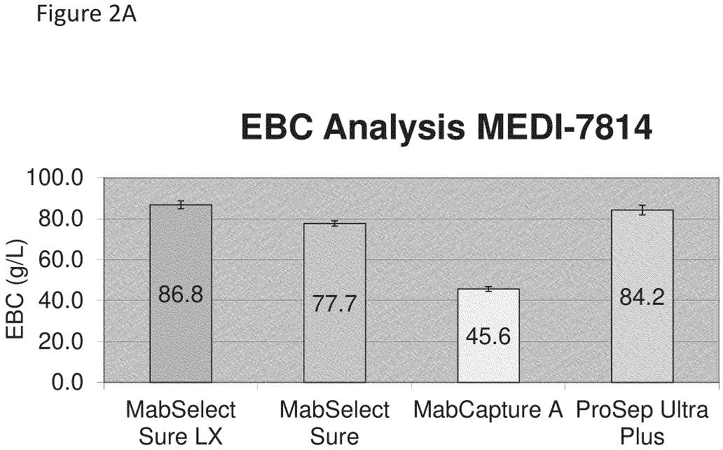

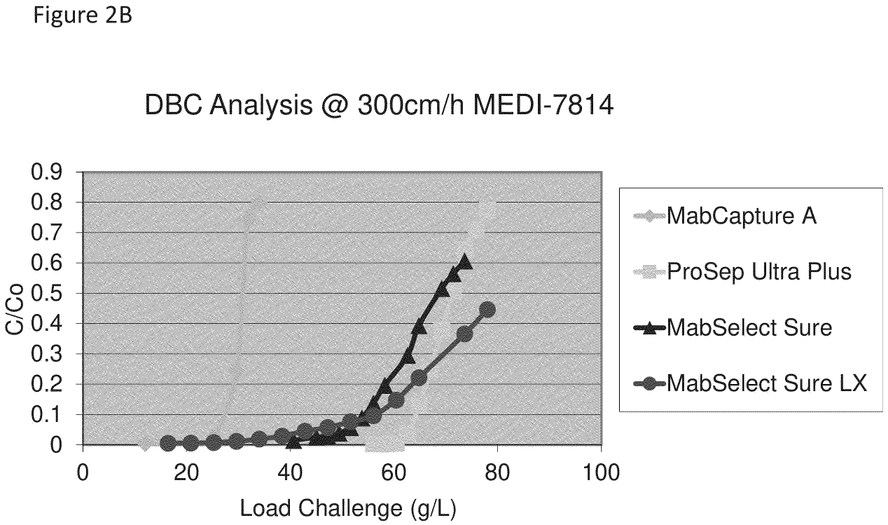

After selecting protein A purification for further purification development, various types of commercially available protein A purification resins were evaluated in the purification process. A binding capacity comparison of MabSelect SuRe, ProSep Ultra Plus, MabCapture A, MabSelect SuRe LX, and AbSolute HiCap was performed using the antibody MEDI-7814 where equilibrium binding capacity (EBC) and dynamic binding capacity (DBC) were compared for each type of Protein A resin. FIG. 2A shows the difference in EBC while FIG. 2B shows the difference in DBC.

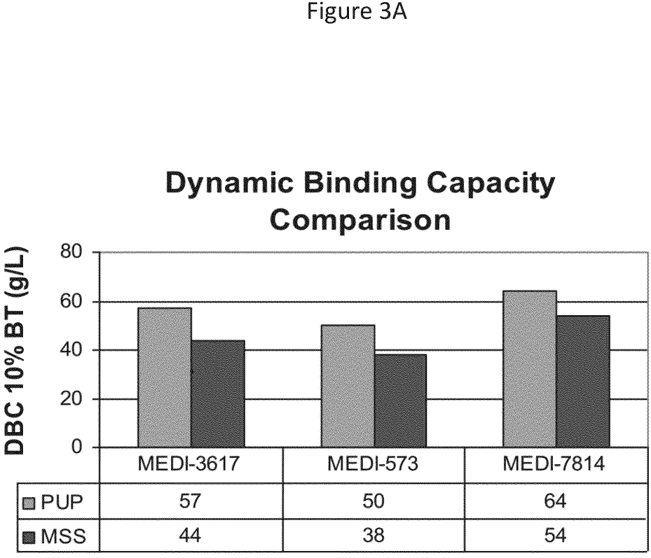

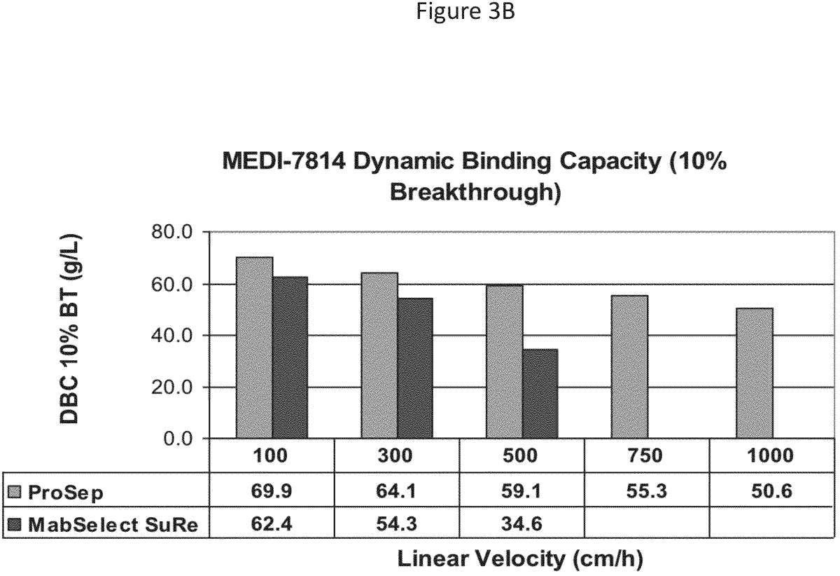

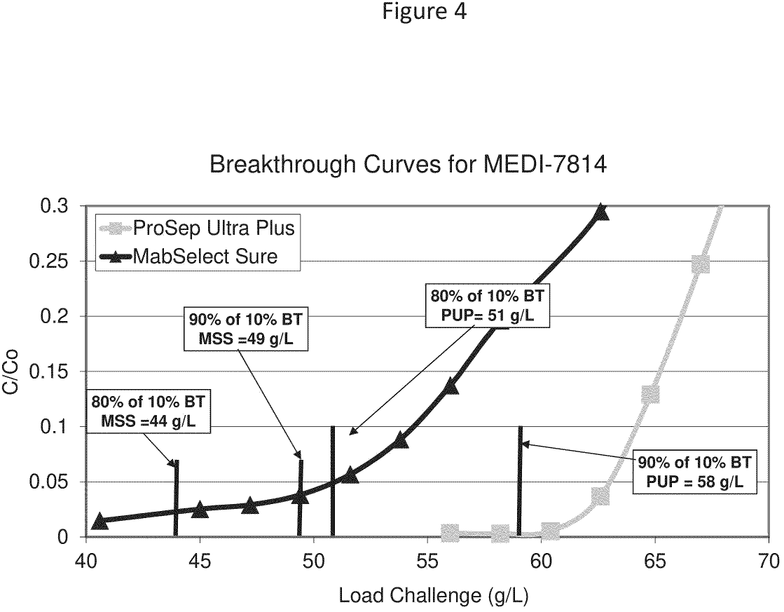

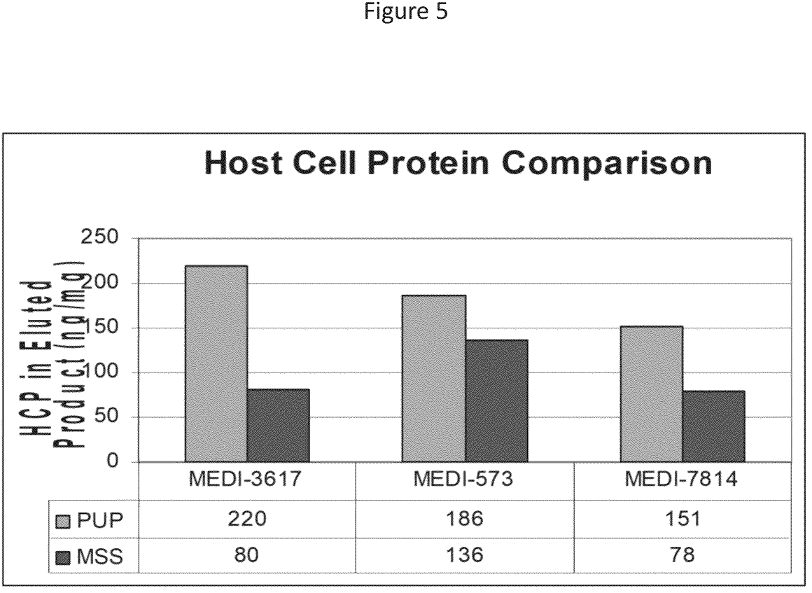

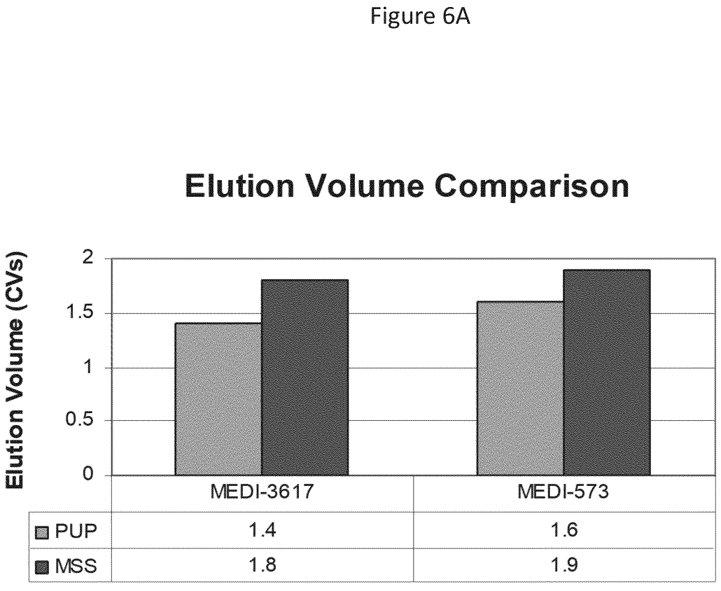

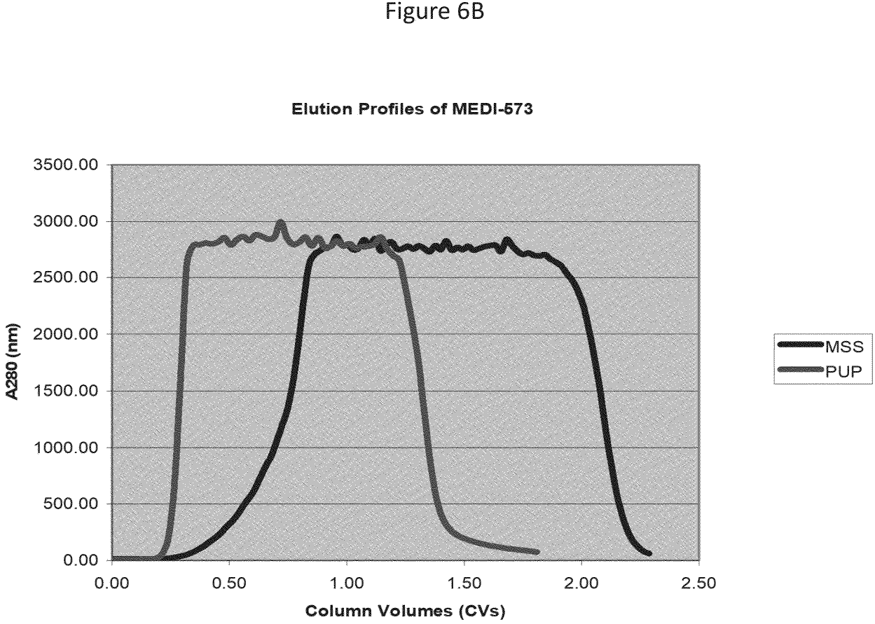

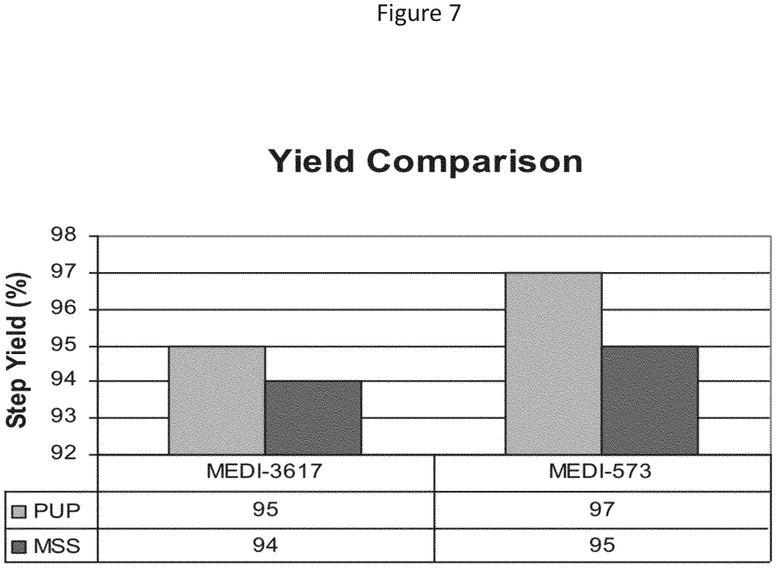

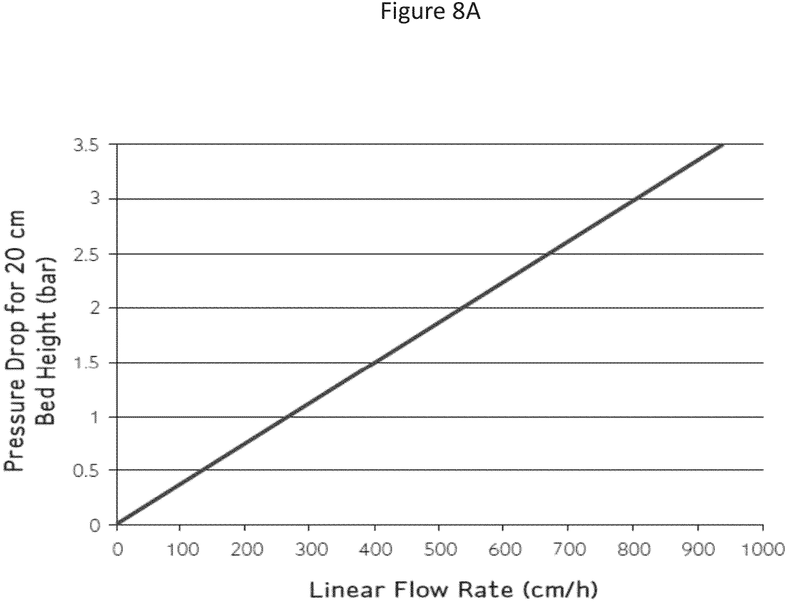

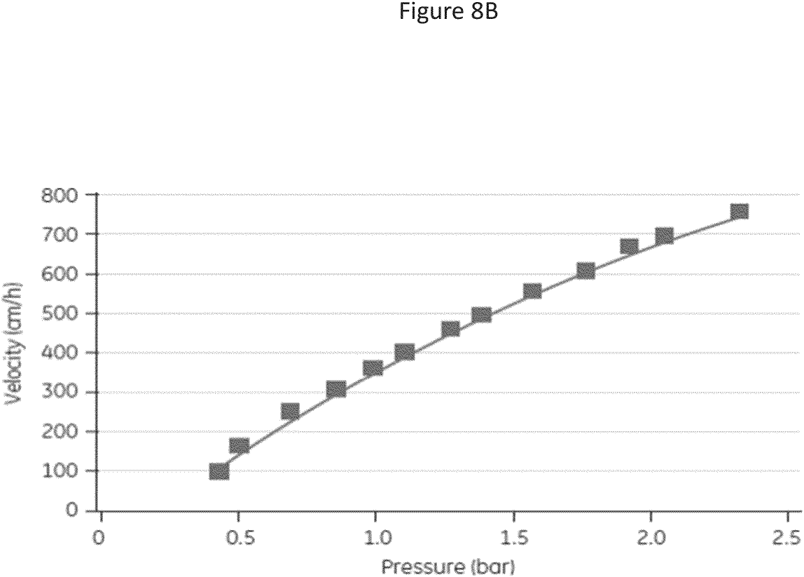

Given the results of the binding capacity, MabSelect SuRe (MSS) and ProSep Ultra Plus (PUP) resins were selected, and a comparison of the two resins was performed with a variety of antibodies using the evaluation criteria of: dynamic binding capacity (FIG. 3A, antibodies MEDI-3617, MEDI-573, and MEDI-7814; FIG. 3B, antibody MEDI-7814), loading capacity safety factor (FIG. 4, antibody MEDI-7814), host cell protein clearance (FIG. 5, antibodies MEDI-3617, MEDI-573, and MEDI-7814), elution volume (FIG. 6A, antibodies MEDI-3617 and MEDI-573; FIG. 6B, antibody MEDI-573), yield (FIG. 7, antibodies MEDI-3617 and MEDI-573), flow properties (FIGS. 8A-8B), cleanability, sanitization buffer, packing method, resin lifetime, and facility fit.

Table 3 summarizes the comparison of MabSelect SuRe and ProSep Ultra Plus resins across the evaluated criteria (+++ = major advantage to that resin; ++ = moderate advantage, + = minor advantage).

TABLE 3

| | | | |
|---|---|---|---|
| Evaluation Criteria | ProSep Ultra Plus (PUP) | MabSelect SuRe (MSS) | Justification |
| DBC | +++ | | PUP has demonstrated increased binding capacity for all projects tested to date. |
| Loading Capacity Safety Factor | ++ | | PUP can be loaded to 90% of 10% breakthrough; MSS can be loaded at 80% of 10% breakthrough. |
| HCP Clearance | | ++ | MSS demonstrated lower HCP in product pools. |
| Elution Volume | ++ | | PUP is able to deliver less product volume. |
| Yield | | | Both resins demonstrate comparable yield. |
| Flow Properties | ++ | | PUP can be operated at higher flowrates (1000 cm/h max). |
| Cleanability | | +++ | Due to the glass backbone, PUP can have cleanability issues. |
| Sanitization Buffer | | + | Hydroxide allows for quicker sanitization. |
| Packing Method | | | No advantage. |
| Resin Lifetime | + | | Millipore claims 300 cycles for resin lifetime while GE Healthcare claims 120 cycles. |
| Facility Fit | +++ | | PUP has comparable or better process fit as compared to MSS. |

Initially, ProSep Ultra Plus Protein A resin was chosen for further process development based on data collected from the comparison as ProSep Ultra Plus showed similar or better process outcomes throughout compared to MabSelect SuRe. In particular, ProSep Ultra Plus allowed for higher dynamic binding capacity, could be operated at higher flowrates without appreciable loss to binding capacity, yielded less product elution volume, and was determined to mitigate potential process bottlenecks. However, due to HCP clearance, and cleanability/sanitization, MabSelect SuRe was ultimately selected as the resin for the purification platform.

In a smaller scale, first-generation purification process, conditioned medium comprising antibody durvalumab was loaded onto a MabSelect SuRe column using a dual flow rate method. Briefly, the column was operated at 350 cm/hour up to 30 mg durvalumab /mL gel and then reduced to 100 cm/hour to reach capacity of 50 mg durvalumab /mL gel. A dynamic capacity study was performed where the conditioned medium was loaded onto the column at a linear velocity of 150 cm/hour to simplify the loading step. The determined capacity at 90% of 10% breakthrough was 57 mg durvalumab /mL gel. A confirmation run performed at 55 mg durvalumab /mL gel showed comparable product quality and yield as illustrated in Table 4.

TABLE 4

| | Yield | DNA (ng/mg) | HCP (ng/mg) | Purity by HPSEC (%) |
|---|---|---|---|---|
| Conditioned Medium | | 2860 | 47326 | |
| Eluate | 88 | 0.51 | 451 | 2.1% aggregate; 97.9% monomer |

### Example 2. Evaluation of a Wash Step

During durvalumab purification process development, high turbidities were seen in some of the MabSelect SuRe eluates. An evaluation was performed to determine if a wash step was able to deal with the turbidities and could also maintain comparable product quality and performance. Previous conditions utilized a wash step of 100 mM Tris, 2.5 M sodium chloride, and 50 mM caprylate at pH 9.0 (hereinafter, “caprylate wash”). The caprylate wash was used with the MabSelect SuRe resin in the commercial purification process and the concentration of host cell protein was measured. Separately, a MabSelect SuRe Protein A column affinity purification was performed without using a caprylate wash and the concentration of host cell protein measured. The use of the caprylate wash resulted in lower host cell protein values; however, the polishing steps later in the process removed the host cell proteins clearing them to comparable levels whether the caprylate wash was performed or not. The HCP concentrations as measured after each step in the process are shown in Table 5.

TABLE 5

| Step | Without Caprylate Wash | With Caprylate Wash |
|---|---|---|
| | HCP (ng/mg) | |
| Conditioned Medium | 42, 197 | |
| MabSelect SuRe Eluate | 378-462 | 67-98 |
| Neutralized low pH Eluate | 460 | 102 |
| Capto Q Flow Through | 104 | 11 |
| HS 50 Eluate | 4 | 3 |

Removing the caprylate high salt wash is desirable due to the tedious preparation on a commercial scale and the overall desire to streamline the process by removing unnecessary buffers. An additional wash evaluation was performed to determine if a wash step was needed and if so, which would be the preferred wash. The evaluation compared washes comprising arginine, NaCl, caprylate, or not including a wash in the affinity chromatography step. The product quality data was comparable between washes, with the caveat of HCP, which shows that including a wash in the process results in lower HCP content in the eluate at this stage of the purification. When NaCl wash was added into the process, the turbidity of the MabSelect SuRe product was reduced. Table 6 summarizes results from the wash step evaluation study.

TABLE 6

| Wash Type | No wash | 58 mM sodium phosphate, 0.5 M arginine, pH 7.0 | 100 mM Tris, 50 mM sodium caprylate, 2.5 M NaCl, pH 9.0 | 52 mM Tris, 1 M NaCl, pH 7.4 |
|---|---|---|---|---|
| Protein load (mg/mL) | 50 | 50 | 50 | 50 |
| Yield (%) | 95 | 87 | 93 | 96 |
| Aggregate (%) | 0.9 | 1.0 | 1.5 | 0.9 |
| Monomer (%) | 99.1 | 99.0 | 98.4 | 98.8 |
| HCP | 208 | 81 | 63 | 143 |

### Example 3. Reduction in Number of Buffers

In an effort to decrease variance and increase the speed of the purification process, experiments were run to identify whether the same buffer could be utilized in more than one step of durvalumab purification. As shown in Table 7, the number of buffers was reduced by using 50 mM Tris, pH 7.4 as the equilibration buffer for the affinity chromatography step and was also implemented as the equilibration buffer in the later anion exchange step. Additionally, the wash buffer for the affinity chromatography step (52 mM Tris, 1 M NaCl, pH 7.4) was not only used as the stripping buffer in both the anion exchange and the cation exchange steps, but was also used for pre-equilibration in the anion exchange step. The purification process also has a low pH treatment step where the acidic titrant is the same buffer as the stripping buffer of the affinity chromatography resin.

TABLE 7

| | |
|---|---|
| Process Step | Buffer |
| Affinity Column Equilibration | 50 mM Tris, pH 7.4 |
| Anion Exchange Equilibration | |
| Affinity Chromatography Wash | 52 mM Tris, 1 M NaCl, pH 7.4 |
| Anion Exchange Equilibration | |
| Cation Exchange Stripping | |
| Affinity Column Stripping | 248 mM glycine, pH 2.35 |
| Low pH Acidic Titrant | |

Another limitation in commercial monoclonal antibody purification is the safety concerns of using 20% ethanol as a column storage buffer. Thus, an alternative storage buffer of 100 mM sodium acetate, 2% benzyl alcohol, pH 5.0 was tested and selected as it did not impact product quality in other similar projects.

### Example 4. Effect on Product Quality of Protein Loading in Affinity Capture Step

The maximum working capacity of MabSelect SuRe was determined to be 50 mg durvalumab /mL gel. Impact of protein loading on affinity column performance studies were conducted. The affinity column was loaded to maximum capacity, half capacity, and maximum capacity at the lowest residence time, where “residence time” represents the amount of time that it takes for a sample to travel the length of a packed chromatography bed. The product quality and column performance were compared for these three conditions. The results (Table 8) show that there is minimal impact of protein load on product quality and process performance.

Table 9 shows parameters for an example embodiment in which MabSelect SuRe is used for antibody capture in the purification process.

TABLE 8

| Load | Yield (%) | Product Volume (CVs) | DNA (ng/mg) | HCP (ng/mg) | Purity by HPSEC (%) |
|---|---|---|---|---|---|
| 50 mg/mL | 89/90 | 1.40 | 0.15 | 137 | 99.2 |
| 25 mg/mL | 80/85 | 1.07 | 0.16 | 122 | 99.3 |
| 50 mg/mL at low residence time | 88/90 | 1.45 | 0.18 | 158 | 99.2 |

TABLE 9

| Step | Buffer | No. of CVs | Linear Flow Rate (cm/hr) |
|---|---|---|---|
| Equilibration | 50 mM Tris, pH 7.4 | ≥3 | 350 |
| Load | | | 150 |
| Wash | 52 mM Tris, 1 M NaCl, pH 7.4 | 5 | 350 |
| Re-equilibration | 52 mM Tris, 1 M NaCl, pH 7.4 | 3 | |
| Elution | 50 mM sodium acetate, pH 3.6 | | |
| Strip | 248 mM Glycine, pH 2.35; 50 mM Glycine, pH 2.3* | 2 | |
| Sanitize | 0.1 N NaOH | 3 | |
| Re-equilibration | 50 mM Tris, pH 7.4 | 3 | |
| Storage | 100 mM sodium acetate, 2% benzyl alcohol, pH 5.0 | 3 | |

### Example 5. Development of Virus Inactivation Step

A virus inactivation step is performed subsequent to the affinity chromatography capture. Virus inactivation commonly occurs through the use of acidic buffers adjusting the pH into an acidic range below pH 4.0 and allowing the titrated pH to remain acidic for over an hour. After holding the titrated product at a pH below 4.0 for the given time period, the titrated product is then neutralized using a basic titrant (acidic and basic titrants are shown in Table 10). The small scale, first-generation durvalumab purification process used an acidic titrant, 100 mM acetic acid to adjust pH of the MabSelect SuRe eluate to pH 3.6 ± 0.1. The acidified product was held for 60 to 90 minutes and then neutralized to pH 5.0 ± 0.2 using 0.5 M Tris base. This resulted in an approximately 100% increase in volume from the affinity eluate. Such an increase in volume poses a constraint on the facility fit. Acidic treatment evaluation was performed, in an effort to reduce the increase in volume, generate a titration curve, and determine the stability.

The target maximum product volume of the affinity chromatography eluate was determined to be 2.2 CVs based on the fit to commercial facility analysis and protein A risk assessment. Additionally, after the low pH treatment step, a 20% increase in volume was determined to be the upper limit of the target.

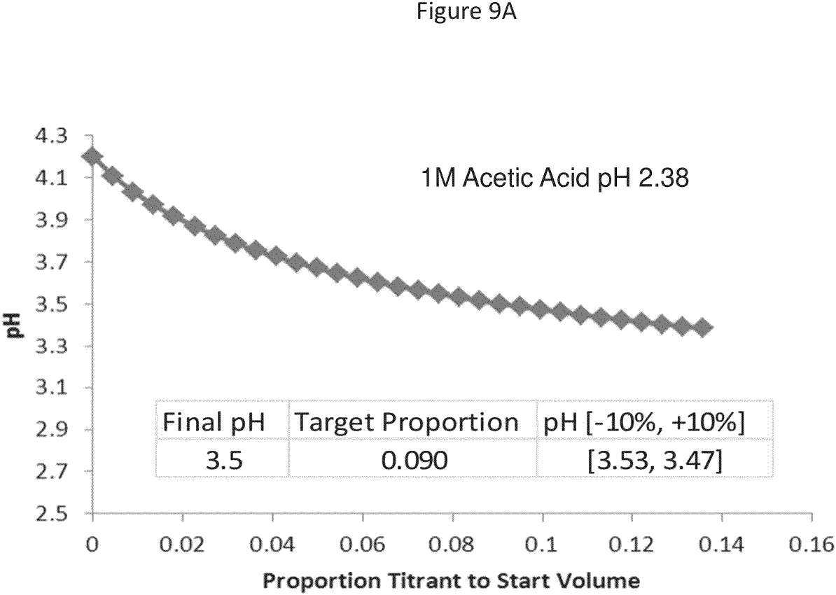

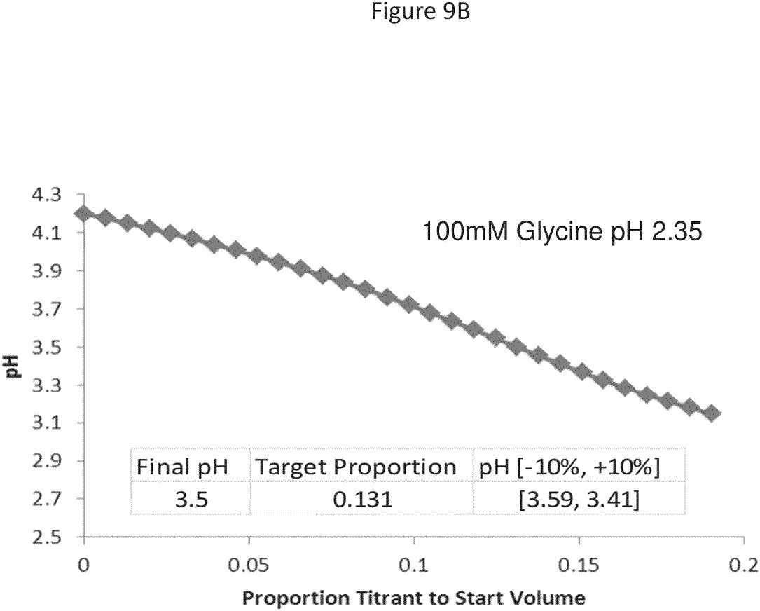

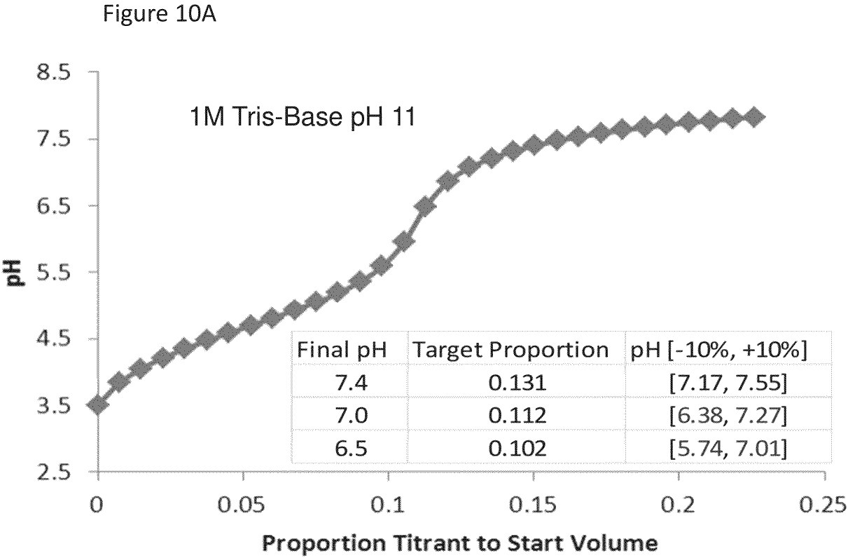

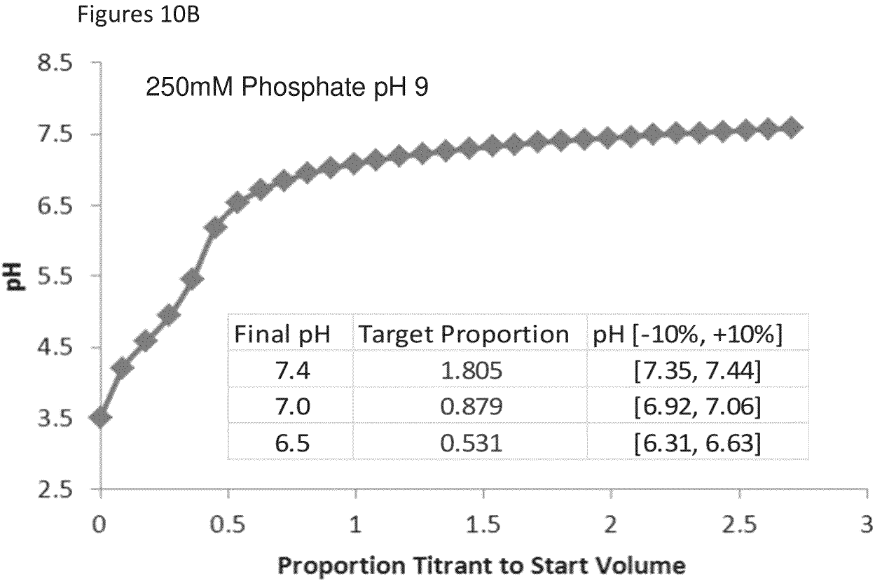

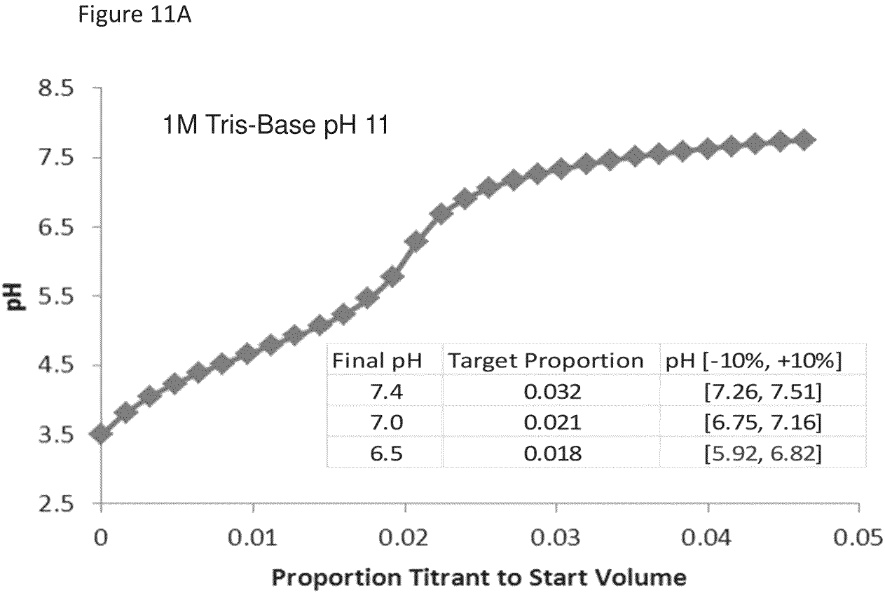

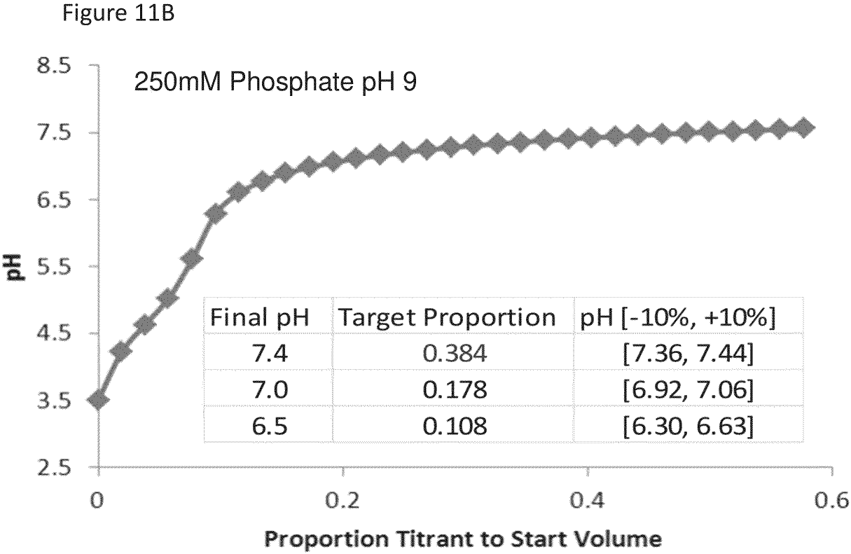

A number of titrants were evaluated as a way to reduce the amount of volume increase in the step and efficiently inactivate viruses during durvalumab purification. The titrants were screened under the assumptions that the low pH step should occur between the affinity chromatography and anion exchange steps, that affinity chromatography was operated with Tris wash buffer and acetate elution buffer, and that the anion exchange operating range supported for viral clearance was pH 6.5 to 7.6 with a target of pH 7.4 ± 0.2. FIGS. 9A-9B represent the acidic titrants of 1 M acetic acid, pH 2.38 compared with 100 mM glycine, pH 2.35 and the proportion of titrant from the start to finish of the pH titration. The eluate titrated to pH 3.5 was then neutralized with either 1 M Tris, pH 11 or 250 mM phosphate, pH 9.0. The results of the neutralization comparison for the eluate titrated with 1 M acetic acid are shown in FIGS. 10A-10B while the results of the neutralization comparison for the eluate titrated with 100 mM glycine are shown in FIGS. 11A-11B.

TABLE 10

| Titrant | | pKa | Notes |
|---|---|---|---|
| Acidic Titrant | Acetic Acid | 4.8 | 1st gen platform |
| | Glycine | 2.4/9.8 | process buffer |
| | Citrate | 3.1 / 4.8 / 6.4 | Undesirable Buffering Range |
| | Phosphate | 2.2 / 7.2 / 12.3 | <pH 2.0 Could Impact Product Quality |
| Basic Titrant | Tris | 8.1 | 1st gen platform |
| | Phosphate | 2.2/7.2/12.3 | process buffer |
| | Bis-Tris | 6.5 | Not Compendial |
| | Citrate | 3.1 / 4.8 / 6.4 | Undesirable Buffering Range |
| | Histidine | 1.7 / 6.0 / 9.1 | Undesirable Buffering Range |

After selecting acetic acid or glycine for the acidic titrant, a number of titrant combinations were evaluated. See Table 11.

TABLE 11

| | | |
|---|---|---|
| | Acidic titrant | Basic titrant |
| 1st generation purification | 100 mM Acetic Acid | 0.5 M Tris Base |
| 2nd generation purification | 100 mM Glycine, pH 2.35 | 1 M Tris |
| | 248 mM Glycine, pH 2.35 | 1 M Tris |
| | 500 mM Glycine, pH 2.35 | 1 M Tris |
| | 248 mM Glycine, pH 2.35 | 0.5 M Tris |

Additionally, the use of acetic acid and glycine as acidic titrants was compared using a number of criteria including dilution factors, total volume of neutralized virus inactivated solution, variability in pH measurement/ease of missing desired pH level, compatible linkage to anion exchange step, shared buffers in purification process, conductivity of neutralized virus inactivated solution, number of chemicals in neutralized virus inactivated solution, ability to reach target pH 5, and leachable compounds from storage container affecting the final product.

TABLE 12

| | | | |
|---|---|---|---|
| Evaluation criterion | 1 M Acetic Acid | 0.25 M Glycine | Notes |
| Dilution Factor (acid) | 22% | 13% | 1 M acetic acid doesn’t work for high titer process. |
| Dilution Factor (base) | 27% | 12% | Flexibility limitation with 1 M acetic acid. |
| Variability In pH Measurement/Ease of Missing Desired pH Level | Yes, for Acidification No, for Neutralization | Yes, for Acidification Yes, for Neutralization | pH sensitive to the addition before reaching the target pH; when overshooting 10%, pH is within 0.2 unit, |
| Compatible Linkage to Anion Exchange Step (Conductivity) | Yes | Yes | N/A |
| Shared Buffer with Affinity Chromatography Stripping | No | No | Can dilute 1 M to 0.1 M acetic acid as ProA strip. |
| Shared Buffer with Cation Exchange Load | Yes | Yes | |
| Number of Chemicals in Neutralized Virus Inactivated Solution, Q, And Cation Exchange | 2 | 3 | 4 for Capto Adhere (0.25 M glycine); may not be a real concern. |
| Target pH 5 | Yes | Yes | Only with phosphate buffer for glycine. |
| Leachable Compounds | Acceptable | Acceptable | Consider leachable Cl from 6 N HCl. |

While acetic acid can be used in the commercial purification of monoclonal antibodies, it was determined that glycine has more benefits as the acidic titrant. Evaluation of the concentration of glycine was performed during durvalumab virus inactivation. The pH set point for the low pH step was pH 3.5, which was in the range of the proposed commercial (second-generation) scheme for monoclonal antibodies. Table 13 shows the results from the glycine titration evaluation study.

TABLE 13

| Sample | Acidic Ratio (mL Acid/ mL Load) | Base Ratio (mL Base/ mL Load) | Total Volume Increase (%) | Yield (%) | Oligomer (%) | Dimer (%) | Monomer (%) |
|---|---|---|---|---|---|---|---|
| MabSelect SuRe | N/A | N/A | N/A | 94 | 0.6 | 0.7 | 98.7 |
| MabSelect SuRe | N/A | N/A | N/A | 92 | 1.0 | 1.2 | 97.5 (0.3% other) |
| 100 mM Glycine, pH 2.35/ 1 M Tris | 0.33 | 0.06 | 39 | 100 | 1.0 | 1.1 | 97.9 |
| 248 mM Glycine, pH 2.35/ 1 M Tris | 0.12 | 0.06 | 15 | 98 | 1.1 | 1.1 | 97.7 |
| 500 mM Glycine, pH 2.35/ 1 M Tris | 0.067 | 0.07 | 9 | 96 | 1.1 | 1.1 | 97.8 |
| 248 mM Glycine, pH 2.35/ 0.5 M Tris | 0.15 | 0.11 | 20 | 99 | 1.1 aggregate | | 98.7 |

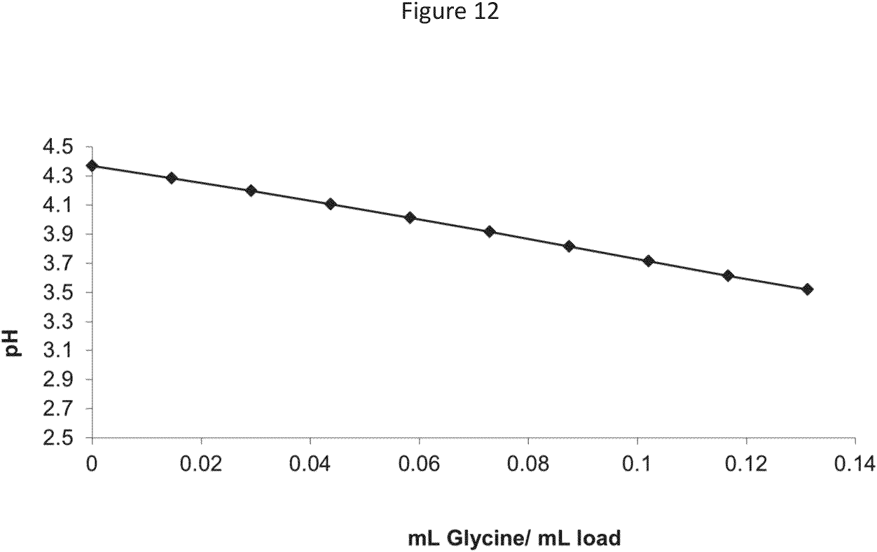

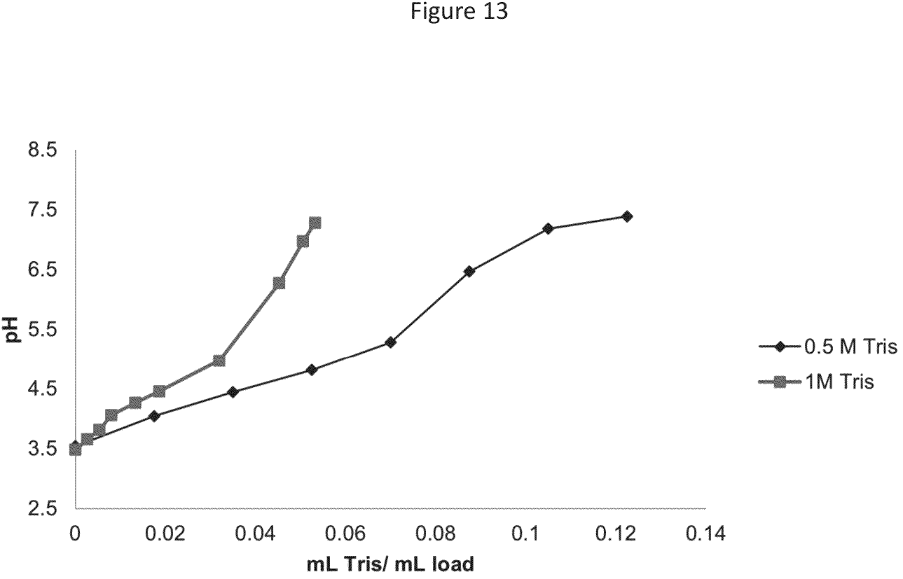

Based on targeting a 20% increase in product volume after the virus inactivation step, it was determined that 248 mM glycine, pH 2.35 for acidic titrant, and 0.5 M Tris as the basic titrant were ideal for the low pH treatment step. Once the titrants were established, titration curves were generated to provide process understanding and to determine the behavior of the durvalumab protein as pH is adjusted. FIG. 12 represents the titration curve for glycine as the acidic titrant. FIG. 13 describes the titration curve comparing basic titrants 0.5 M Tris and 1 M Tris during the neutralization portion of the low pH treatment step.

Due to the desired limitation of preferring a 10% increase in volume after each titrant addition, 0.5 M Tris buffer is recommended because this solution gives a ratio of 0.11 mL/mL load.

### Example 6. Evaluation of Low pH Treatment on Product Quality

In the first-generation durvalumab purification process, the hold time at acidic pH was 60 to 90 minutes. To understand product quality and process impact outside of manufacturing range, a study was performed holding the acidified product at 0.1 pH unit below worst case (pH 3.3) for up to 24 hours before neutralizing the material. The turbidity and purity of the neutralized low pH treated product was analyzed and compared to a control.

After the acidified products were neutralized, the samples were analyzed for purity by HPSEC, and the turbidity was measured in Nephelometric Turbidity Units (NTU). Table 14 shows the results of the analysis, demonstrating no observable impact on product quality at longer hold times at pH 3.3. Increasing hold time was proportional to an increase in turbidity, likely due to co-precipitation of non-product related impurities.

TABLE 14

| Acidic Hold Time | Purity by HPSEC (%) | | | Turbidity (NTU) | Yield (%) |
|---|---|---|---|---|---|
| | Aggregate | Monomer | Other | | |
| 1 hour | 0.9 | 99.0 | 0.1 | 110 | 88 |
| 6 hours | 0.6 | 99.3 | 0.1 | 125 | 85 |
| 24 hours | 0.9 | 99.0 | 0.1 | 140 | 83 |

The filters typically used to filter process intermediates are a nominal 0.2 µm filter, which can handle solutions up to 10 NTU. As shown in Table 14, the turbidity values measured were >10 NTU, suggesting the need of a depth filter such as POD filter before the 0.2 µm filter. In some embodiments, the POD filter C0HC (Millipore) was used for filtering neutralized material prior to loading onto the anion exchange chromatography step. Yields were lower than expected due to sampling of the material for the turbidity measurements not allowing for accurate volume measurement.

### Example 7. Development of Anion Exchange Chromatography Step

The anion exchange step in the antibody purification platform is intended to remove impurities such as DNA and putative viruses. Contrary to the sequence of polishing chromatography steps in a first-generation purification process, the second-generation platform uses the anion exchange as the first polishing step. The two anion exchange chromatography modes studied included a resin-based chromatography, Capto Q, and membrane chromatography, Mustang Q.

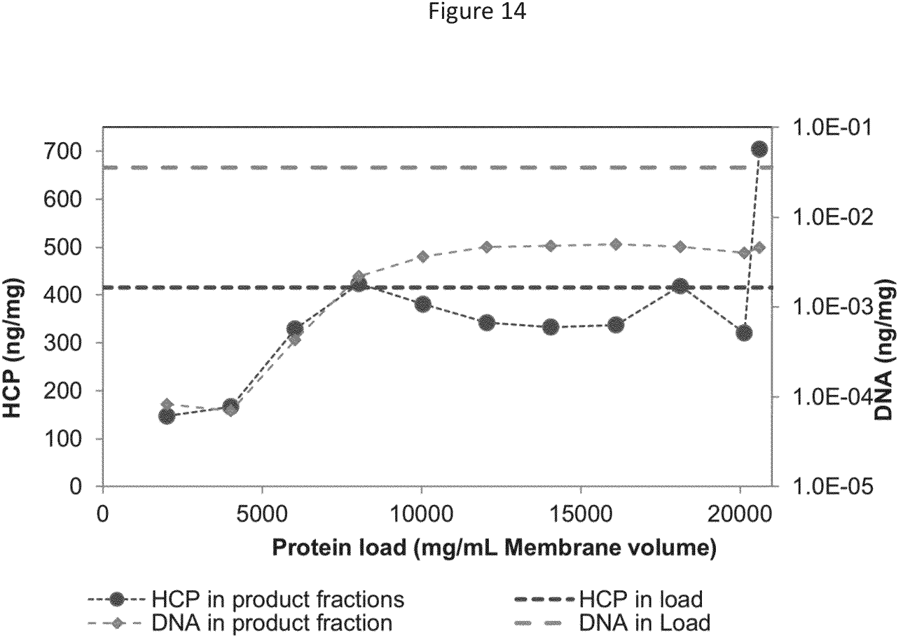

In an effort to improve throughput, anion exchange membrane chromatography was evaluated. A process fit analysis using BioFit was performed for the Mustang Q anion exchange membrane and suggested that an increase in load capacity from 5 g/L membrane to 20 g/L membrane would result in a reduction of the number of cycles. The impact of increasing protein load on the membrane was tested. The load material, neutralized low pH treated product, was loaded onto the Q membrane to a capacity of 20 g durvalumab /mL membrane volume. The eluate was fractionated and collected every 2,000 mg durvalumab /mL membrane volumes and analyzed for HCP, DNA, and yield. The breakthrough curves for HCP and DNA resulting from the Mustang Q membrane study are shown in FIG. 14.

Based on these data, a 5,000 mg durvalumab /mL membrane volume was selected for operation of the Q membrane due to breakthrough of HCP and DNA observed beyond this point.

Previous applications perform the anion chromatography step at 100 mg/mL gel. Due to the desire of reducing the overall process time, a reduction in the number of cycles for the anion chromatography was sought. The impact of protein load on Capto Q was evaluated by loading the column to 75 mg durvalumab /mL gel versus 150 mg durvalumab /mL gel. The eluate fractions for each separate experiment were collected and analyzed for step yield, purity by HPSEC, and HCP. Table 15 summarizes the results of the protein load study.

TABLE 15

| | Neutralized Low pH Treated Product (pH 7.4) | 75 ng durvalumab /mL Load Product | 150 ng durvalumab/mL Load Product |
|---|---|---|---|
| Step yield (%) | N/A | 94.2 | 98.0 |
| Aggregate by HPSEC (%) | 0.4 (multimer) 0.4 (dimer) | 0.5 (dimer) | 0.5 (dimer) |
| Monomer by HPSEC (%) | 99.2 | 99.5 | 99.5 |
| HCP (ng/mg) | 46 | 10 | 7 |

The data suggest that there is no difference in product quality when the Capto Q chromatography step is loaded to 75 versus 150 mg durvalumab /mL gel. Since operating the Capto Q step at 150 mg durvalumab /mL gel would require fewer cycles, throughput of the purification process would be increased as compared to 75 durvalumab /mL gel loading.

Additionally, as seen in Table 15, the Capto Q step removed multimers in addition to HCPs. Table 16 shows an embodiment of the Capto Q step in the purification process.

TABLE 16

| Step | Buffer | No. of CVs | Linear Flow Rate (cm/hour) | Collection |
|---|---|---|---|---|
| Pre-equilibration | 52 mM Tris, 1 M NaCl, pH 7.4 | ≥3 | 350 | |
| Equilibration | 50 mM Tris, pH 7.4 | ≥4 | | |
| Load | | | | <_ 150 mg/mL; start at 0.5 OD |
| Chase | 50 mM Tris, pH 7.4 | | | End at 0.5 OD |
| Strip | 52 mM Tris, 1 M NaCl, pH 7.4 | ≥2 | | |
| Sanitization | 1 N NaOH | ≥2 | | |
| Storage | 0.1 N NaOH | ≥2 | | |

### Example 8. Development of Cation Exchange Step

The cation exchange chromatography step is the final polishing step of the antibody purification process and is used in a bind and elute mode. Three main goals were kept in mind while developing the cation exchange step: increasing productivity (product bound/cycle), addressing facility fit concerns (number of CVs/cycle), and providing for robust clearance of aggregates.

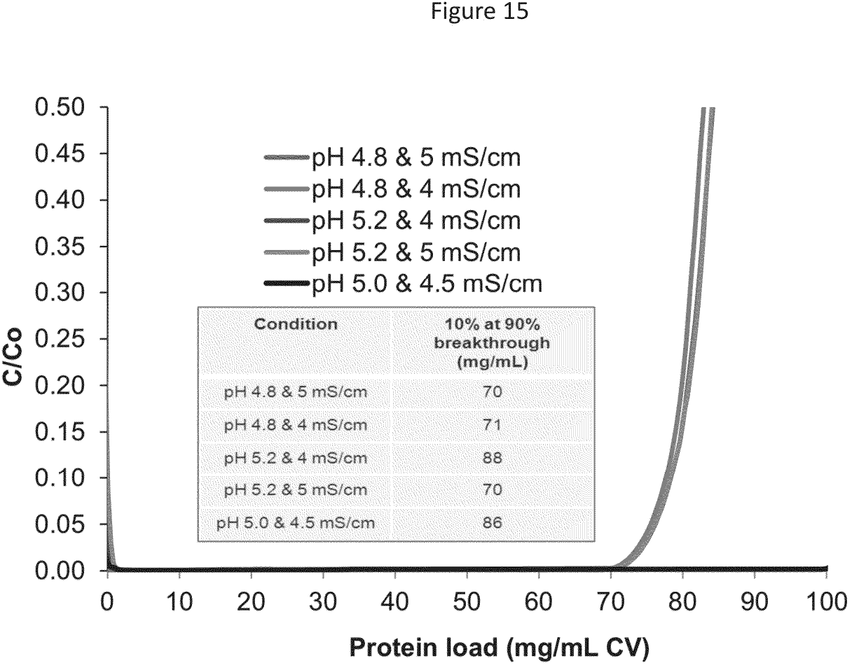

Poros HS 50 was evaluated as the final step for the second-generation protein purification process. Use of Poros HS 50 as the polishing step after anion exchange chromatography requires adjusting the pH of the load material to more acidic pH ranges. Optimization of dynamic binding capacity was conducted with a full factorial design with one center point; a study was performed using neutralized low pH-treated durvalumab product that was adjusted to pH range of 4.8 to 5.2 and conductivity of 4.0 to 5.0 mS/cm. FIG. 15 shows the graph of the DBC curves for the conditions and Table 17 shows the 10% at 90% breakthrough numbers.

TABLE 17

| Condition | 10% at 90% Breakthrough (mg/mL) |
|---|---|
| pH 4.8 & 5 mS/cm | 70 |
| pH 4.8 & 4 mS/cm | 71 |
| pH 5.2 & 4 mS/cm | 88 |
| pH 5.2 & 5 mS/cm | 70 |
| pH 5.0 & 4.5 mS/cm | 86 |

The dynamic binding capacities were consistent at the conditions tested (≥70 mg durvalumab /mL gel) at 100 cm/hour. The maximum binding capacity for the second-generation process was set to 60 mg durvalumab /mL gel, which increased from 50 mg durvalumab /mL gel in the first-generation process. The increase in the binding capacity was obtained by reducing the flow rate during loading from 200 to 100 cm/hour.

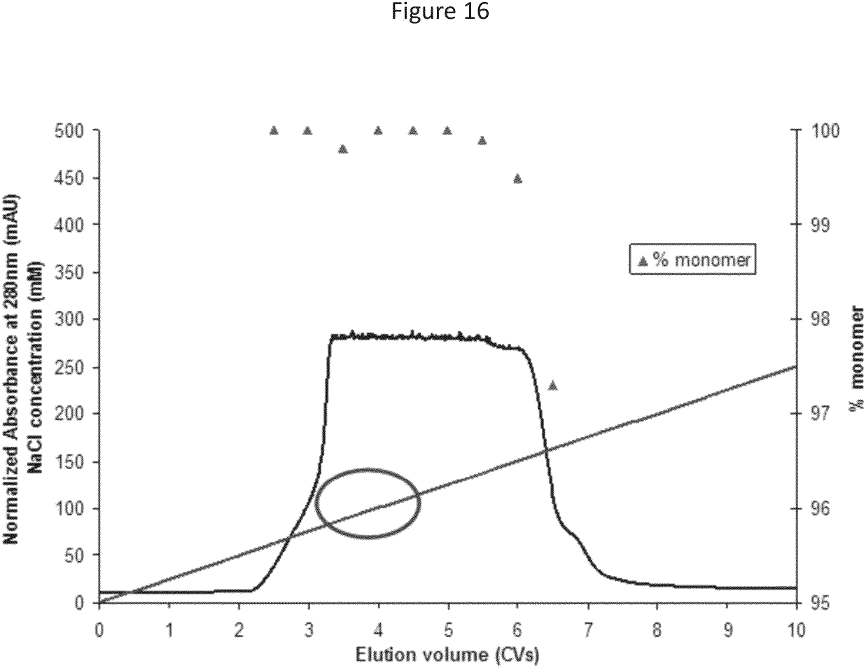

To streamline the elution step of cation exchange chromatography in the purification process, various elution buffers were examined to determine the optimal eluting condition for impurity removal. A linear gradient experiment was performed where neutralized low pH-treated durvalumab product adjusted to pH 5.0 was loaded to 60 mg durvalumab /mL gel. The linear gradient went from 20 mM Histidine/ Histidine-HCl, pH 6.0 to 20 mM Histidine/ Histidine-HCl, 500 mM NaCl, pH 6.0, and eluate fractions were collected and analyzed for purity by HPSEC and step yield. Results from this study are shown in FIG. 16.

Based on the results, elution conditions ranging from 115 to 150 mM NaCl proved to be the most promising because product quality was similar or better when compared to the smaller scale purification process. To determine the optimal condition within that range, a more in depth study was executed where step elution experiments were performed, and the eluates were analyzed for product quality and column performance (Table 18).

TABLE 18

| Elution Buffer | Product Volume (CVs) | Yield (%) | Purity by HPSEC (%) | | | HCP (ng/mg) |
|---|---|---|---|---|---|---|
| | | | Aggregate | Monomer | Other | |
| 75 mM NaCl, pH 6.9 (1st gen.) | 2.6 | 88 | 0.1 | 99.7 | 0.1 | 27 |
| 115 mM NaCl, pH 6.0 | 2.4 | 88 | 0.1 | 99.8 | | 23 |
| 125 mM NaCl, pH 6.0 | 1.7 | 87 | 0.5 | 99.4 | 0.1 | 35 |
| 130 mM NaCl, pH 6.0 | 1.7 | 91 | 0.6 | 99.4 | | 31 |
| 150 mM NaCl, pH 6.0 | 1.7 | 89 | 0.6 | 99.4 | | 45 |

The elution conditions that were evaluated produced similar step recovery and HCP content in the eluate; however, some differences in product volume and purity were evident. The 115 mM NaCl condition was in line with the smaller scale purification process results in terms of product volume and purity. The elution condition selected was 20 mM Histidine/ Histidine-HCl, pH 6.0 where operating at lower pH may provide better viral clearance.

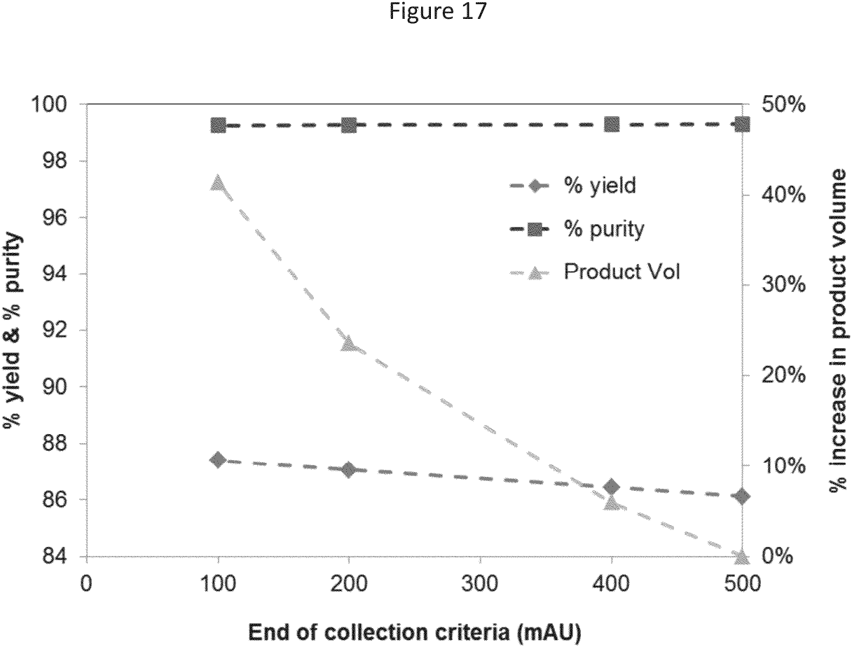

Since improvements in yield and product quality are desired when scaling to the second-generation purification process, yield and product quality were evaluated using neutralized low pH-treated durvalumab products adjusted to pH 5.0 and loaded into the cation exchange column at 60 mg durvalumab /mL gel. The column was eluted using the buffer of 20 mM Histidine/ Histidine-HCl, 115 mM NaCl, pH 6.0. Eluate fractions were collected and analyzed for step recovery, purity by HPSEC, DNA, and HCP. FIG. 17 shows the results where the fractions collected were: main peak to 2.5 OD (500 mAU), 2.5 to 2.0 OD (500 mAU to 400 mAU), 2.0 to 1.0 OD (400 mAU to 200 mAU), and 1.0 to 0.5 OD (200 mAU to 100 mAU).

As shown in FIG. 17, there was no noticeable change to purity and step yield as more protein was eluted off the column; however, there was an increase in product volume. This increase did not result in a significant impact on the clearance of aggregate or an increase in cation exchange chromatography step yield.

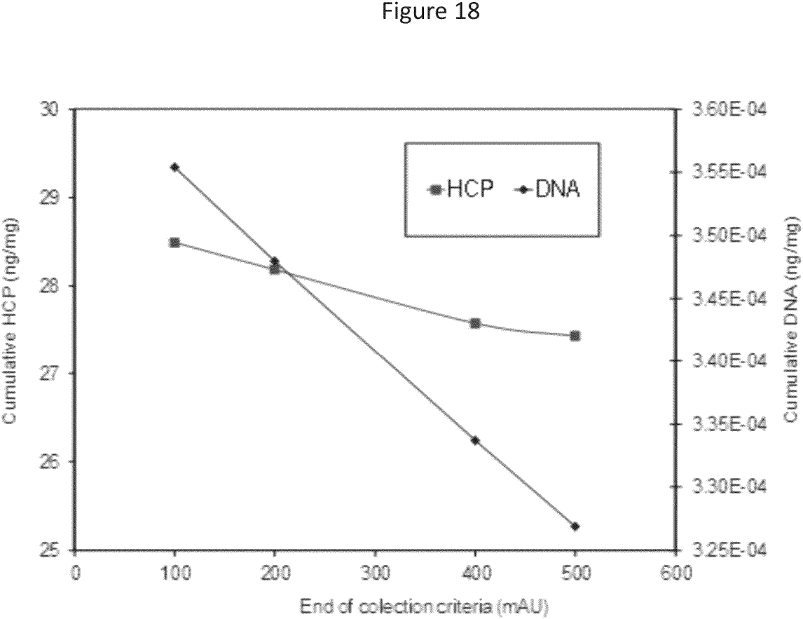

FIG. 18 shows the HCP and DNA content results from the durvalumab HS 50 fractionation study. Although an increase is shown in HCP and DNA after 500 mAU, the increase was not significant. For HCP the difference was 25 to 29 ng/mg and for DNA going from 3.25 x 10⁻⁴ to 3.55 x 10⁻⁴ ng/mg. Therefore, the end elution cut off criterion was determined to be 500 mAU (2.5 OD).

The first-generation durvalumab purification process was run with all the steps of cation exchange chromatography at a flow rate of 200 cm/hour, the only exception being the elution step in which the flow rate was reduced to 100 cm/hour. The elution flow rate was decreased due to possible pressure spikes that may have occurred because of high concentration of protein as it was eluting from the column. To further improve throughput, a study was conducted to examine the effect of increasing the flow rate in all parts of the chromatography steps except the loading step. The study was performed to see how increasing the flow rate from 200 to 300 cm/hour would impact column performance. The experiment involved performing two experiments operated at 200 versus 300 cm/hour, except the loading step was performed at 60 mg durvalumab /mL gel using neutralized low pH treated product adjusted to pH 5.0. The eluates were collected and compared for product quality and column performance; data is shown in Table 18.

TABLE 18

| Flow Rate | Yield (%) | Purity by HPSEC (%) | Aggregate by HPSEC (%) | HCP (ng/mg) | DNA (ng/mg) |
|---|---|---|---|---|---|
| HS 50 Load | - | 98.6 | 1.6 | 311 | 1.91 ×10⁻² |
| 200 cm/hour | 88 | 99.8 | 0.2 | 24 | 1.75 x 10⁻⁴ |
| 300 cm/hour | 89 | 99.8 | 0.2 | 30 | 1.13 ×10⁻⁴ |

The data in Table 19 show that the cation exchange chromatography step, performed with HS 50, can be operated at a flow rate of 300 cm/hour for all steps except the load without any impact on product quality.

TABLE 19

| Step | Buffer | No. of CVs | Linear flow rate (cm/hour) |
|---|---|---|---|
| Equilibration | 50 mM Sodium Acetate, pH 5.0 | ≥3.0 | 300 |
| Load (≤ 60 mg/mL) | | | 100 |
| Wash | 26 mM Histidine/ Histidine-HCl, pH 5.8 | ≥3.0 | 300 |
| Elution (0.5-2.5 OD Collection) | 20 mM Histidine/ Histidine-HCl, 115 mM NaCl, pH 6.0 | | |
| Strip | 52 mM Tris, 1 M NaCl, pH 5.4 | ≥2.0 | |
| Sanitize | 1 N NaOH | ≥3.0 | |
| Storage | 0.1 N NaOH | ≥3.0 | |

### Example 9. Determination of Polishing Steps Sequence

The order of the polishing steps can be interchanged, meaning anion exchange chromatography can be performed before the cation exchange chromatography or cation exchange chromatography can be performed before anion exchange chromatography. Due to this variation in order, the polishing step order was evaluated based on a number of factors including product quality consistently achieved, impurity and viral clearance capability, volumetric throughput up to 10 g/L, minimization of process differences between monoclonal antibodies, and simplicity in development and operation. The process of anion exchange chromatography before cation exchange chromatography showed major advantages in terms of product quality consistently achieved, impurity and viral clearance capability, accommodation of high titer processes, and minimization of process differences between monoclonal antibodies (data not shown). On the other hand, cation exchange chromatography before anion exchange chromatography showed a slight advantage in simplicity of operation.

### Example 10. Development of Filtration Steps

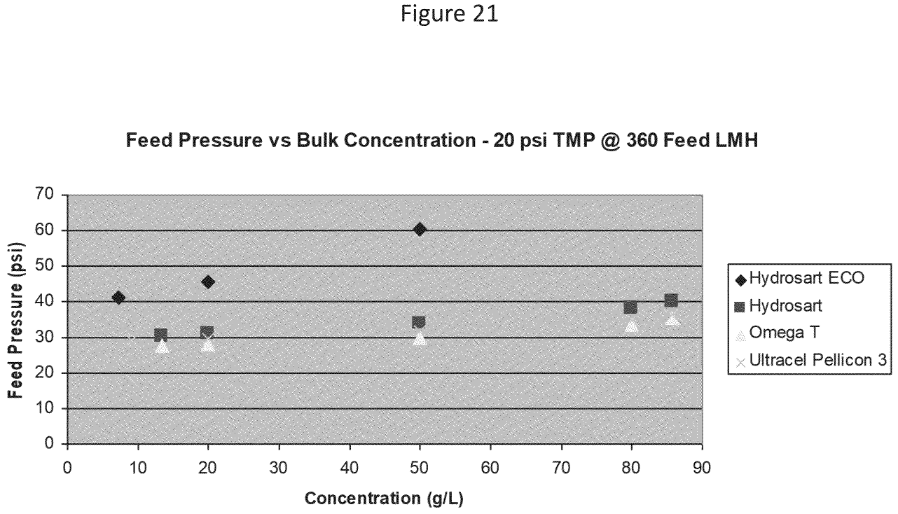

Virus filtration steps follow the polishing steps of commercial protein purifications. Different types of filtration can be used, and it was determined that in order to streamline the durvalumab purification process, the ultrafiltration/diafiltration platform should meet the need for both low (20 mg/ml) and high (≥150 mg/mL) formulations. The ultrafiltration/diafiltration platform was evaluated on criteria including product quality criteria consistently achieved, minimization of resources required, process performance criteria achieved consistently, footprint in manufacturing plant, and ease in cleaning. All membranes evaluated were commercially obtained and vendor support was also included in evaluation criteria (Hydrosart ECO, Hydrosart, Pall Omega T, and Pellicon 3 Ultracell). A comparison of feed pressure versus bulk concentration of the four membranes is shown in FIG. 21. While all membranes were functional, the performance of Millipore Pellicon 3 Ultracell was preferable across the most evaluation criteria (Table 20). Additionally, Pall Omega PES T-Series membranes gave positive results across many of the evaluation criteria.

TABLE 20

| | Water Permeability (LMH/psi) | Mass Transfer Coeff., K, (LMH) | Calculated C_{wall}(g/L) | Highest concentration (g/L) | Average Flux (50-90 g/L) (LMH) |
|---|---|---|---|---|---|
| Omega-T | 13.1 | 36.9 @ TMP = 20 psi | 128.2 | 85.7 | 24.2 |
| P3 Ultracell RC, C screen | 9.6 | 33.2 @ TMP = 20 psi | 115.8 | 85.7 | 19.4 |
| Hydrosart | 5.3 | 27.5 @ TMP = 25 psi | 154.3 | 85.7 | 9.9 |
| Hydrosart ECO | 7.1 | 32.4 @ TMP = 25 psi | 140.2 | 50 | 10.8 |

### Example 11. Bench Top Scale Purification Run for anti-PD-L1 Monoclonal Antibody

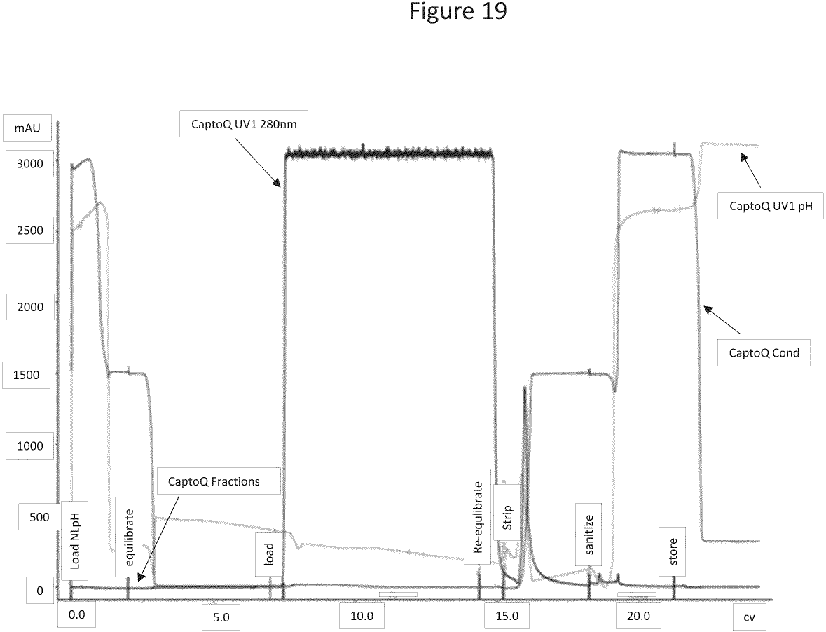

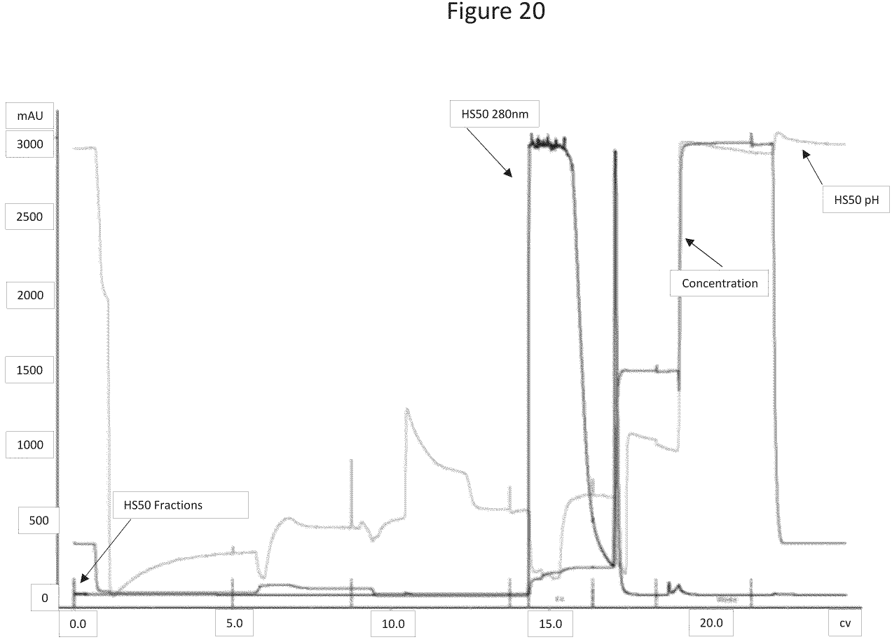

Following purification process development, an experimental run was performed of the entire purification process as selected during development. While the purification platform is contemplated for use with a wide variety of antibodies, the experimental run was performed for purification of anti-PD-L1 monoclonal antibody durvalumab. Product quality and performance were measured and compared after each process step. Table 21 shows the analysis results of the bench top scale purification after each process step. The chromatograms from the bench top scale purification run for the anion exchange chromatography and cation exchange chromatography steps are shown in FIGS. 19 and 20, respectively.

TABLE 21

| Process Step | Yield (%) | Purity by HPSEC (%) | | | DNA (ng/mg) | HCP (ng/mg) |
|---|---|---|---|---|---|---|
| | | Aggregate | Monomer | Other | | |
| MabSelect SuRe | 99-101 | 0.7-0.9 | 98.8-99.3 | 0-0.3 | 6.88 x 10⁻²- 9.19 x 10⁻² | 134-173 |
| Low pH | 95 | 1.1 | 98.7 | 0.1 | 5.79 ×10⁻⁴ | 90 |
| Capto Q | 103 | 1.1 | 98.5 | 0.3 | < 5.77E x 10⁻⁴ - <5.85 x 10⁻ 4 | 10-12 |
| HS 50 | 89-91 | 0.5-0.6 | 99.2-99.5 | 0.2 | 1.83 ×10⁻⁴- <5.16 x 10⁻⁵ | <3 - <5 |
| Nanofiltration | 98-99 | 0.5 | 99.2-99.3 | 0.2 | Not tested | Not tested |
| UF/DF | 106 | 0.6 | 99.1 | 0.2 | <5.80 ×10⁻⁵ | <1.5 |

Table 22 shows the purification process steps for an example embodiment and the example conditions that are included with each step. The conditions marked with an asterisk (*) are optional.

TABLE 22

| | |
|---|---|
| Step/Process Operation | Conditions |
| MabSelect SuRe Affinity Capture | Load rate of 150 cm/hr up to 50 mg/mL durvalumab |
| | Wash* with 52 mM Tris, 1 M NaCl, pH 7.4 |
| | Strip* with 50 mM Glycine, pH 2.3 |
| | Store* column in 100 mM sodium acetate, 2% benzyl alcohol, pH 5.0 |
| Low pH Treatment Virus Inactivation | Titrate to pH 3.5 ± 0.1 using 248 mM Glycine, pH 2.35 |
| | Hold time at low pH: 45 - 240 min |
| | Neutralize to pH 7.4 ± 0.2 using 0.5 M Tris |
| Polishing Step ⅟Anion Exchange Chromatography | Capto Q step operated at capacity of≤ 150 mg/mL |
| | Pre-equilibration buffer* same as MabSelect SuRe wash buffer and HS 50 strip buffer |
| | Chase with 50 mM Tris, pH 7.4 |
| | Strip* with 52 mM Tris, 1 M NaCl, pH 7.4 |
| | Sanitize* with 1 N NaOH |
| | Store* in 0.1 N NaOH |
| Polishing Step 2/ Cation Exchange Chromatography | Poros HS 50 |
| | Load flow rate at 100 cm/hour at capacity of ≤60 mg/mL |
| | All other flow rates at 300 cm/hour |
| | Elution buffer: 20 mM Histidine/ Histidine-HCl, |
| | 115 mM NaCl, pH 6.0; end elution criteria is 2.5 OD |
| | Strip* with 52 mM Tris, 1 M NaCl, pH 7.4 |
| | Sanitize* with 1 N NaOH |
| | Store* in 0.1 N NaOH |
| Virus Filtration | vPro+ (Millipore) |
| Diafiltration/Ultrafiltration | P3 Ultracell RC, C screen |

### Example 12. N-Linked Oligosaccharide Identification

The Fc region of durvalumab contains an N-linked oligosaccharide chain attached to a single site on the heavy chain at Asn-301. Structural characterization of the oligosaccharides on durvalumab is critical to the understanding of the structural micro heterogeneity of the product. It is also important for quality control when the process changes.

The oligosaccharides cleaved from durvalumab that were present in the 2-AB labeled N-linked oligosaccharides profile by ultra-performance liquid chromatography (UPLC) were characterized, including the low abundance glycoforms.

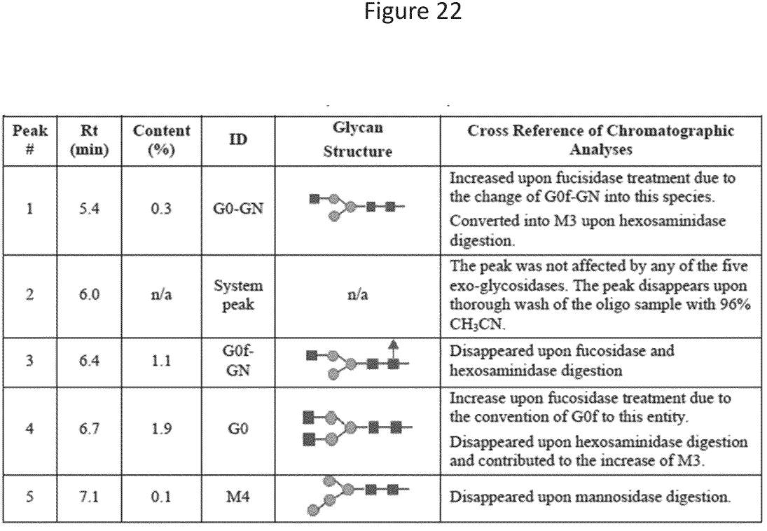

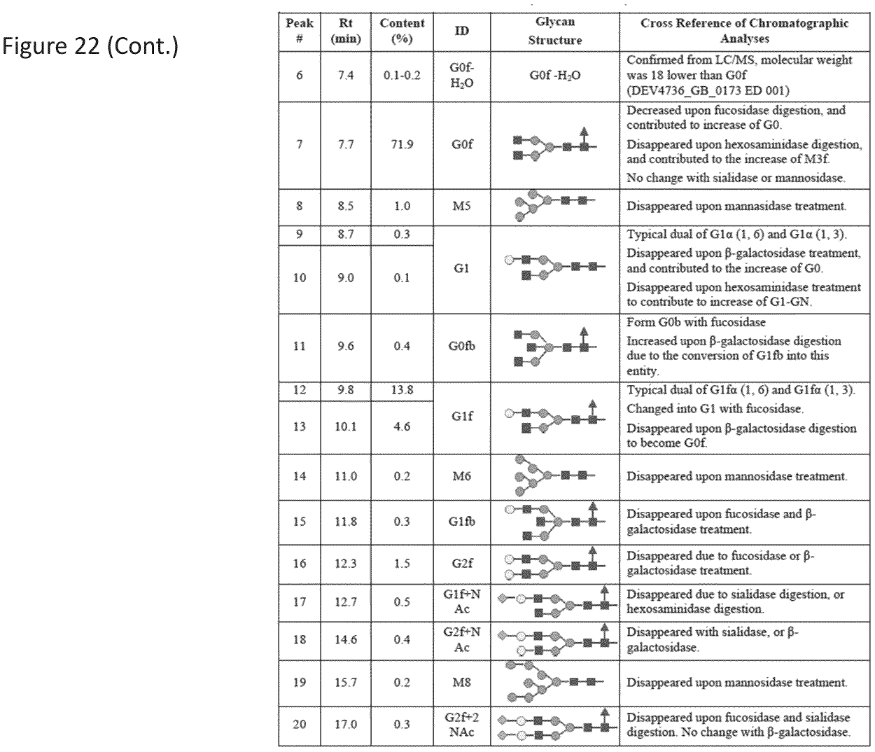

The N-linked oligosaccharides in durvalumab that were detected as significant peaks in UPLC were identified and the results are shown in FIG. 22. The predominant glycoforms of durvalumab are fucosylated biatennary complex-type oligosaccharides with either no terminal galactose residues (GOf), or mono-galactosylated (Glf), and di-galactosylated (G2f) forms. The minor complex glycoforms were afucosylated G0and G1, truncated G0f and G0 forms without N-acetyl-glucosamine (GlcNAc), sialylated G1f and G2f forms (G1f+NAc, G2f+NAc, or G2f+2NAc), and bisecting structures G0fb and G1fb. Depending on the maturity of the Chinese Hamster Ovary (CHO) cell when IgG was harvested, high mannose glyocforms (Man4, Man5, Man6, Man7, and Man8) were also present.

### Example 13. Analytical Analysis of Purified Durvalumab Preparations

After formulation, purified durvalumab preparations can be analyzed by capillary isolectric focusing (cIEF). For example, samples can be adjusted to 0.25 mg/mL with HPLC grade water. Samples can then be digested with Carboxypeptidase B (CBP) for 10 minutes at 37° C. then diluted with 1% methylcellulose solution, Pharmalyte pH 3 - 10, pI marker 9.46, and pI Marker 5.85. Samples can then be loaded onto an iCE280 Analyzer and focused at 1500 V for 1 minute, followed by 3000 V for 7 minutes. The resulting electropherograms can then be analyzed using EZChrom software and compared to a reference standard.

After formulation, melting temperature, and isoelectric point (pI) of purified durvalumab preparations can be determined. Melting temperature of purified durvalumab preparations can be determined using Differential Scanning Calorimetry (DSC). For example, the pI and stability of durvalumab can be determined using capillary isoelectric focusing and high-pressure size exclusion chromatography (HPSEC), respectively.

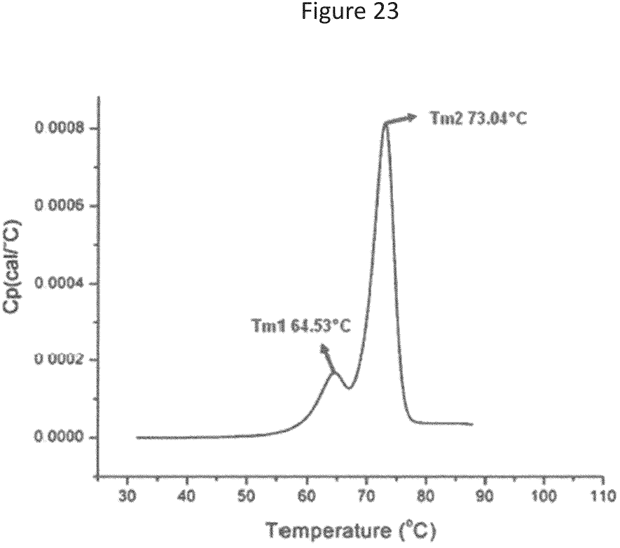

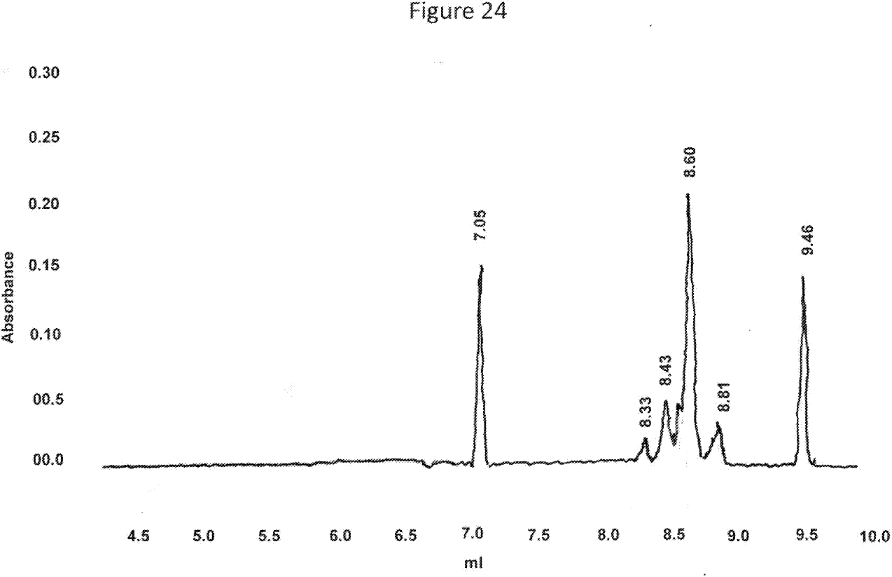

An example of the DSC profile of purified and formulated durvalumab was shown as Tm1 at 64.5° C. and Tm2 at 73.04° C. (FIG. 23). Four peaks, ranging from 8.3 - 8.8 were detected in the capillary Isoelectric focusing (cIEF) profile of durvalumab. The main peak had an isoelectric point of 8.6 (FIG. 24).

While the disclosure has been described in terms of various embodiments, it is understood that variations and modifications will occur to those skilled in the art. Therefore, it is intended that the appended claims cover all such equivalent variations that come within the scope of the disclosure as claimed. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

1. A method of purifying an antibody, the method comprising:
(a) subjecting a sample comprising the antibody to affinity chromatography to capture the antibody and eluting the antibody from the affinity chromatography column to produce an eluate comprising the antibody, wherein the affinity chromatography column is washed with a washing buffer comprising 4.0 M or lower sodium chloride at pH 6.0 to pH 8.0;
(b) treating the eluate from step (a) by adding an amount of a one or more solutions that reduces or increases the pH of the eluate to produce a neutralized eluate comprising the antibody;
(c) subjecting the neutralized eluate from step (b) to anion exchange chromatography and collecting a flow-through product comprising the antibody;
(d) subjecting the flow-through product from step (c) to cation exchange chromatography and eluting the antibody from the cation exchange chromatography column to produce a cation exchange chromatography product comprising the antibody;
(e) subjecting the cation exchange chromatography product from step (d) to virus filtration to produce a virus filtration product comprising the antibody; and
(f) subjecting the virus filtration product from step (e) to ultrafiltration to recover the purified antibody.

2. The method of claim 1, wherein the affinity chromatography column is a protein-A based resin column.

3. (canceled)

4. The method of claim 1, wherein the step of subjecting the sample comprising the antibody to affinity chromatography comprises:
(i) equilibrating the affinity chromatography column with an equilibrating buffer at pH 6.0 to pH 8.0;
(ii) re-equilibrating the affinity chromatography column with the equilibrating buffer at pH 6.0 to pH 8.0; and
(iii) eluting the antibody from the affinity chromatography column using an elution buffer at pH 3.0 to pH 4.0.

5. The method of claim 4, wherein the equilibrating buffer comprises 50 mM Tris at pH 7.4.

6. The method of claim 4, wherein the elution buffer comprises 50 mM sodium acetate at pH 3.6.

7. The method of claim 4, further comprising washing the affinity chromatography column following step (i) and prior to step (ii).

8. (canceled)

9. The method of claim 1, wherein the washing buffer comprises 52 mM Tris and 1.0 M sodium chloride at pH 7.4.

10. The method of claim 1, wherein the step of treating the eluate to produce a neutralized eluate comprising the antibody comprises:
(i) titrating the eluate to a pH range of pH 3.0 to pH 5.0 using an acidic titrant solution;
(ii) incubating the eluate and acidic titrant solution in the titrated pH range for at least 15 minutes; and
(iii) neutralizing the eluate and acidic titrant solution to neutral pH using a basic titrant solution.

11. The method of claim 10, wherein the acidic titrant solution comprises 100 mM glycine to 500 mM glycine at pH 2.0 to pH 3.0 and the basic titrant solution comprises 0.5 M Tris to 1.0 M Tris.

12. The method of claim 11, wherein the acidic titrant solution comprises 248 mM glycine at pH 2.35 and the basic titrant solution comprises 0.5 M Tris.

13. The method of claim 1, further comprising filtering the neutralized eluate of step (b) through at least one 0.2 µm filter prior to subjecting the neutralized eluate to anion exchange chromatography.

14. The method of claim 1, further comprising filtering the neutralized eluate of step (b) through a depth filter and then through a 0.2 µm filter prior to subjecting the neutralized eluate to the anion exchange chromatography.

15. The method of claim 1, wherein the step of subjecting the neutralized eluate to anion exchange chromatography is performed using a resin-based chromatography membrane.

16. The method of claim 15, wherein the anion exchange chromatography is performed using Capto a Q resin.

17. The method of claim 15, wherein the anion exchange chromatography is performed using a Q membrane.

18. The method of claim 1, wherein the neutralized eluate from step (b) comprises up to 500 mg/mL of the antibody when subjected to anion exchange chromatography.

19. The method of claim 1, wherein the step of subjecting the neutralized eluate to anion exchange chromatography and collecting a flow-through product comprising the antibody comprises:
(i) equilibrating the anion exchange chromatography column with an equilibrating buffer at pH 6.0 to pH 8.0;
(iii) passing the neutralized eluate through the anion exchange chromatography column; and
(iv) collecting the flow-through product comprising the antibody from the anion exchange chromatography column.

20. The method of claim 19, further comprising chasing the neutralized eluate with a chase solution at pH 3.0 to pH 4.0 and collecting the chase solution that flows through the anion exchange chromatography column to produce a flow-through product comprising the antibody.

21. The method of claim 1, wherein the cation exchange chromatography is performed using a resin-based column.

22. (canceled)

23. The method of claim 1, wherein the step of subjecting the flow-through product comprising the antibody to a cation exchange chromatography comprises:
(i) equilibrating the cation exchange chromatography column with a cation exchange equilibrium buffer at pH 4.5 to pH 5.5;
(ii) loading up to 100 mg/mL of flow-through product onto the cation exchange chromatography column;
(iii) washing the cation exchange chromatography column with a cation exchange washing buffer at pH 5.0 to pH 7.0; and
(iv) eluting a cation exchange chromatography product comprising the antibody from the cation exchange chromatography column using a cation exchange elution buffer at pH 5.0 to pH 7.0.

24. The method of claim 23, wherein the cation exchange equilibrium buffer comprises 50 mM sodium acetate at pH 5.0, the cation exchange washing buffer comprises 26 mM Histidine/Histidine-HCl at pH 5.8, and the cation exchange elution buffer comprises 20 mM Histidine/Histidine-HCl and 115 mM sodium chloride at pH 6.0.

25. The method of claim 1, wherein the step of subjecting the cation exchange chromatography product to virus filtration is performed using a virus filtration membrane.

26. The method of claim 1, wherein the step of subjecting the virus filtration product to ultrafiltration is performed using a TFF membrane.

27. The method of claim 1, wherein the purified antibody is a human anti-PD-L1 antibody.

28. The method of claim 27, wherein the human anti-PD-L1 antibody comprises a light chain region comprising the amino acid sequence of SEQ ID NO: 1 and a heavy chain region comprising the amino acid sequence of SEQ ID NO: 2.

29. The method of claim 27, wherein the human anti-PD-L1 antibody comprises the amino acid sequences of SEQ ID NOs: 3-8.

30. The method of claim 1, wherein the purified antibody recovered in step (f) constitutes 60% to 70% of the antibody in the sample comprising the antibody.

31. The method of claim 1, wherein at least 99% of the purified antibody recovered in step (f) is present as a monomer as measured by high pressure size exclusion chromatography (HP-SEC).

32. The method of claim 1, wherein the DNA content of the purified antibody recovered in step (f) is less than 0.2 pg/mg.

33. The method of claim 1, wherein less than 1% of the purified antibody recovered in step (f) forms an aggregate as measured by high pressure size exclusion chromatography (HP-SPEC).

34. The method of claim 1, wherein the host cell protein content of the purified antibody recovered in step (f) is less than 10 ng/mg.

35. The method of claim 1,
wherein the antibody preparation comprises a main form of the antibody comprising greater than, or equal to, 45% of the protein in the composition as measured using capillary isoelectric focusing (cIEF), acidic forms of the antibody comprising 45% to 50% of the protein in the composition as measured using cIEF, and a basic form of the antibody comprising 18% to 23% of the protein in the composition as measured using cIEF.

36. The method of claim 1,
wherein 1.5% to 2.5% of the antibody in the antibody preparation forms an aggregate as determined by high-pressure size exclusion chromatography (HP-SPEC); and wherein 97% to 98% of the antibody in the antibody preparation is present as a monomer as measured by HP-SEC.

37. The method of claim 1,
wherein the glycan structures of the purified antibody preparation comprise G0f, G1f, G2f, and G0 glycoforms.

38. The method of claim 37, wherein the glycan structures of the purified antibody preparation have a content greater than 90% for the G0f, G1f, G2f, and G0 forms.

39. The method of claim 37, wherein the purified antibody preparation comprises 71.9% G0f content, 18.4% G1f content, 1.5% G2f content, and 1.9% G0 content.

40-43. (canceled)

44. The method of claim 1, wherein the washing buffer further comprises 52 mM Tris.

45. The method of claim 1, wherein the washing buffer is at pH 7.4.


SEQUENCE LISTING (8 sequences)

SEQ ID NO: 1 - protein, 108 aa, synthetic construct
  REGION 1..108: note = light chain (VL)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, synthetic construct
  REGION 1..121: note = heavy chain (VH)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, synthetic construct
  REGION 1..10: note = CDR1-H
GFTFSRYWMS

SEQ ID NO: 4 - protein, 17 aa, synthetic construct
  REGION 1..17: note = CDR2-H
NIKQDGSEKYYVDSVKG

SEQ ID NO: 5 - protein, 12 aa, synthetic construct
  REGION 1..12: note = CDR3-H
EGGWFGELAFDY

SEQ ID NO: 6 - protein, 12 aa, synthetic construct
  REGION 1..12: note = CDR1-L
RASQRVSSSYLA

SEQ ID NO: 7 - protein, 7 aa, synthetic construct
  REGION 1..7: note = CDR2-L
DASSRAT

SEQ ID NO: 8 - protein, 9 aa, synthetic construct
  REGION 1..9: note = CDR3-L
QQYGSLPWT